(19) United States
(12) Patent Application Publication
(10) Pub. No.: US 2023/0190802 A1
(43) Pub. Date: Jun. 22, 2023

(54) METHOD OF TREATMENT OF CANCER OR TUMOUR

(71) Applicant: ADAPTIMMUNE LIMITED, Abingdon, Oxfordshire (GB)

(72) Inventor: Mark Edward DUDLEY, Abingdon, Oxfordshire (GB)

(21) Appl. No.: 17/924,980

(22) PCT Filed: May 13, 2021
(86) PCT No.: PCT/GB2021/051158

§ 371 Date: Jan. 23, 2023

Related U.S. Application Data
(60) Provisional application No. 63/024,104, filed on May 13, 2020.

(51) Int. Cl. A61K 35/17; C07K 14/725; A61P 35/00

(52) U.S. Cl. CPC A61K 35/17; C07K 14/7051; A61P 35/00

(57) ABSTRACT

The present invention relates to a method of treating, preventing or delaying the progression of cancer and/or tumour in a subject comprising administering to the subject a treatment regimen comprising an effective amount of modified immunoresponsive cells expressing or presenting a heterologous T-cell receptor (TCR) having the property of binding to MAGE A4

### FIELD OF INVENTION

The present invention relates to a method of treating, preventing or delaying the progression of cancer and/or tumour in a subject comprising administering to the subject a treatment regimen comprising an effective amount of modified immunoresponsive cells expressing or presenting a heterologous T-cell receptor (TCR) having the property of binding to MAGE A4 or antigenic peptide thereof, in particular the treatment of gastroesophageal cancer.

### BACKGROUND

Gastroesophageal cancer and/or tumour encompasses cancer and/or tumour of the esophagus, gastro-esophageal junction or stomach, i.e. gastric cancer.

Esophageal cancer: Esophageal cancer is the sixth most common cause of cancer-related death worldwide. Generally esophageal cancers arise from the epithelium of the esophagus and falls into one of two classes: esophageal squamous-cell carcinomas (ESCC), which are strongly linked with tobacco and alcohol consumption, and esophageal adenocarcinomas (EAC), commonly associated with of GERD and Barrett's esophagus. Esophageal cancer includes esophagogastric junction cancer or carcinoma or adenocarcinoma (EGJ) which is a cancer of the lower part of the esophagus, often linked to a Barrett's esophagus. EGJ is a highly mutated and heterogeneous disease with an elevated number of somatic mutations across a number of genes for example in CR2, HGF, FGFR4, ESRRB, TP53, SYNE1, and ARID1A. Treatment options for esophagogastric junction adenocarcinomas are limited and the overall prognosis is extremely poor. ESCC (esophageal squamous-cell carcinoma) accounts for 60-70% of all cases of esophageal cancer worldwide, a further 20-30% of cases are EAC (esophageal adenocarcinoma), other less common forms of the cancer include melanomas, leiomyosarcomas, carcinoids and lymphomas. In general, the prognosis of esophageal cancer is quite poor, the overall five-year survival rate in the United States is around 15%, with most people dying within the first year of diagnosis. Recent data for England and Wales show that only ten percent of people survive esophageal cancer for at least ten years. Poor prognosis accounts for the prevalence of the disease, poor survival is also linked to low rates of early detection as most patients present with advanced disease by the time the first symptoms such as difficulty swallowing appear. In the United States, esophageal cancer is the seventh-leading cause of cancer death among males. Curative treatment options for the localised disease combine surgery, radiation and chemotherapy.

Metastatic or recurrent disease is commonly managed palliatively by radiation and chemotherapy with stenting to relieve symptoms and make it easier to swallow.

Gastric and stomach cancer: Gastric and stomach cancer are closely linked to tobacco, alcohol and H. pylori, and eating salted or pickled foods. Treatment generally includes a surgical combination with chemotherapy (by drugs such as 5-fluorouracil, cisplatin, epirubicin, etoposide, docetaxel, oxaliplatin, capecitabine or irinotecan), radiation therapy or targeted therapy, e.g. treatment with human epidermal growth factor receptor 2 (HER2) inhibitor, trastuzumab, HER2 is overexpressed in 13-22% of patients with gastric cancer. Although some types of gastric lymphoma can be cured by eliminating H. pylori, generally outcomes are often poor, largely because most people with the condition present with advanced disease. Globally, stomach cancer is the third leading cause of death from cancer and occurs twice as often in males as in females, making up 9% of deaths, in the United States, five-year survival is 31.5%.

It is desirable therefore to provide a therapy for tumour and/or cancer treatment, such as treatment of gastroesophageal cancer and/or tumour, including cancer and/or tumour of the esophagus, gastro-esophageal junction or gastric cancer and/or tumour, wherein the therapy is cancer specific, capable of treating intermediate or late stage cancer or single or multiple solid tumours, particularly where there has been failure or recurrence following primary therapy or surgery, preferably also where the therapy minimises or reduces toxicity or side effects for example risk of systemic toxicity of chemotherapeutic agents (e.g. nausea, vomiting, anaemia, and thrombocytopenia) or tissue damage due to radiotherapy.

The present invention relates to and exemplifies the treatment of gastroesophageal cancer and/or tumour in a subject comprising administering to the subject a treatment regimen comprising an effective amount of modified T-cells expressing or presenting a heterologous T-cell receptor (TCR) having the property of binding to MAGE A4 and in particular, specifically binding to GVYDGREHTV, SEQ ID NO: 2. In particular, the HLA-A2 restricted MAGE A4 peptide GVYDGREHTV, SEQ ID NO: 2, which provides a suitable target for novel immunotherapeutic interventions; this peptide is naturally processed and has been isolated from gastroesophageal cancer cell lines.

### SUMMARY OF THE INVENTION

According to a first aspect of the present invention there is provided a method of treating, preventing or delaying the progression of gastroesophageal cancer and/or tumour in a subject comprising administering to the subject a treatment regimen comprising an effective amount of modified immunoresponsive cells expressing or presenting a heterologous T-cell receptor (TCR) or chimeric antigen receptor (CAR) binding to MAGE A4 or a MAGE A4 antigenic peptide thereof.

According to the invention the TCR or CAR may bind MAGE A4, or an antigenic peptide thereof, for example Human MAGE A4 or MAGE A4 of SEQ ID NO: 1 or an antigenic peptide thereof. The TCR or CAR may bind to an antigenic peptide comprising SEQ ID NO: 2, GVYDGREHTV.

In one aspect, the invention provides a modified immunoresponsive cell expressing or presenting a heterologous T-cell receptor (TCR) or CAR which binds a peptide antigen of MAGE A4 or an antigenic peptide thereof, for use in treating, preventing or delaying the progression of cancer and/or tumour in a subject, wherein the cancer and/or tumour is gastroesophageal cancer and/or tumour.

In embodiments, the TCR or CAR may bind Human MAGE A4 or MAGE A4 of SEQ ID NO: 1 or an antigenic peptide thereof. The TCR or CAR may bind to an antigenic peptide comprising SEQ ID NO: 2, GVYDGREHTV.

In embodiments, said cell is administered to a subject in a treatment regimen comprising an effective amount of said cells.

Immunoresponsive Cells

According to the present invention the modified immunoresponsive cells can be cells of the lymphoid lineage, comprising B, T or natural killer (NK) cells. The modified immunoresponsive cells may be cells of the lymphoid lineage including T cells, Natural Killer T (NKT) cells, and precursors thereof including embryonic stem cells, and pluripotent stem cells (e.g, those from which lymphoid cells may be differentiated). T cells can be lymphocytes that mature in the thymus and are chiefly responsible for cell-mediated immunity and also involved in the adaptive immune system. According to the present invention the T cells can include, but are not limited to, helper T cells, cytotoxic T cells, memory T cells (including central memory T cells, stem-cell-like memory T cells (or stem-like memory T cells), and two types of effector memory T cells: e.g. TEM cells and TEMRA cells, Regulatory T cells (also known as suppressor T cells), Natural Killer T cells, Mucosal associated invariant T cells, and gamma-delta T cells. Cytotoxic T cells (CTL or killer T cells) are a subset of T-lymphocytes capable of inducing the death of infected somatic or tumour cells. A subject's own T cells may be genetically modified to target specific antigens through the introduction of a heterologous TCR or CAR. Preferably, the modified immunoresponsive cell is a T cell optionally a CD4⁺ T cell or a CD8⁺ T cell. Accordingly the modified immunoresponsive cells may be T-cells, optionally CD4+ T cells or CD8+ T cells, or the modified immunoresponsive cells may be a population of modified T-cells, optionally CD4+ T cells; or CD8+ T cells, or a mixed population of CD4+ T cells and CD8+ T cells.

Heterologous TCR/CAR

According to the present invention the modified immunoresponsive cells can express a heterologous T cell receptor (TCR) or heterologous chimeric antigen receptor (CAR) (e.g., the cell is transduced with or engineered to comprise a nucleic acid sequence encoding a heterologous TCR or CAR, for example by gene knock in). Upon binding to the antigen, the modified immunoresponsive cells can exhibit T cell effector functions and/or cytolytic effects towards cells bearing the antigen and/or undergo proliferation and/or cell division. In certain embodiments, the modified immunoresponsive cells comprising the heterologous TCR exhibits comparable or better therapeutic potency compared to cells comprising a chimeric antigen receptor (CAR) targeting the same cancer and/or tumour antigen and/or peptide (antigenic peptide). Activated modified immunoresponsive cells comprising the heterologous TCR or CAR can secrete anti-tumour cytokines which can include, but are not limited to, TNFalpha, IFNy and IL2.

According to the invention the modified immunoresponsive cells may comprise a nucleic acid, construct or vector, or heterologous nucleic acid, construct or vector, encoding a heterologous T cell receptor (TCR) or heterologous chimeric antigen receptor (CAR). Optionally the TCR may be an affinity enhanced TCR, for example a specific peptide enhanced affinity receptor (SPEAR) TCR.

The term “heterologous” or “exogenous” refers to a polypeptide or nucleic acid that is foreign to a particular biological system, such as a cell or host cell, and is not naturally present in that system and which may be introduced to the system by artificial or recombinant means. Accordingly, the expression of a TCR or CAR which is heterologous, may thereby alter the immunogenic specificity of the immunogenic cells, for example T cells, so that they recognise or display improved recognition for one or more tumour or cancer antigens and/or peptides that are present on the surface of the cancer cells of an individual with cancer. The modification of immunogenic cells or T cells and their subsequent expansion may be performed in vitro and/or ex vivo.

Cancer/Tumour Antigen or Peptide Antigen

According to the present invention the cancer and/or tumour antigen or peptide antigen thereof may be a cancer-testis antigen, such as a MAGE, melanoma associated antigen or member of the MAGEA gene family, for example any one of MAGE A1, A2, A3, A4, A5, A6, A7, A8, A9, A10, A11, or A12, or peptide antigen thereof. Preferably the tumour antigen is MAGE-A4, SEQ ID No:1, or peptide antigen thereof. Preferably the cancer and/or tumour antigen peptide comprises or has the amino acid sequence GVYDGREHTV, SEQ ID NO: 2.

Co-Stimulatory Ligand

According to the present invention, the modified immunoresponsive cells, may further comprise an exogenous or a recombinant (e.g., the cell is transduced with or engineered to comprise a nucleic acid sequence encoding a co-stimulatory ligand, for example by gene knock in) at least one co-stimulatory ligand, optionally one, two, three or four. The modified immunoresponsive cells, may co-express the heterologous TCR or CAR and the at least one exogenous or heterologous co-stimulatory ligand. The interaction between the heterologous TCR or CAR and at the least one exogenous co-stimulatory ligand may provide a non-antigen-specific signal and/or activation of the cell. Co-stimulatory ligands include, but are not limited to, members of the tumour necrosis factor (TNF) superfamily, and immunoglobulin (Ig) superfamily ligands. TNF is a cytokine involved in systemic inflammation and stimulates the acute phase reaction. TNF superfamily members include, but are not limited to, nerve growth factor (NGF), CD40L (CD40L)/CD154, CD137L/4-1BBL, TNF-alpha, CD134L/OX40L/CD252, CD27L/CD70, Fas ligand (FasL), CD30L/CD153, tumour necrosis factor beta (TNFP)/Iymphotoxin-alpha (LTa), Iymphotoxin-beta (TTb), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins— they possess an immunoglobulin domain (fold). Immunoglobulin superfamily ligands include, but are not limited to, CD80 and CD86, both ligands for CD28. In certain embodiments, the at least one co-stimulatory ligand is selected from the group consisting of 4-1BBL, CD275, CD80, CD86, CD70, OX40L, CD48, TNFRSF14, and combinations thereof. The at least one exogenous or recombinant co-stimulatory ligand can be 4-1 BBL or CD80, preferably, the at least one exogenous or recombinant co-stimulatory ligand is 4-1 BBL. The modified immunoresponsive cells may comprise two exogenous or recombinant co-stimulatory ligands, preferably the two exogenous or recombinant co-stimulatory ligands are 4-1 BBL and CD80.

The modified immunoresponsive cells may comprise an exogenous or a recombinant (e.g., the cell is transduced with or engineered to express, for example by gene knock in) at least one construct which overcomes the immunosuppressive tumour microenvironment. Such constructs can be, but are not limited to, cyclic AMP phosphodiesterases and dominant-negative transforming growth factor beta (TGFbeta) receptor II. The modified immunoresponsive cell, modified T cell or a population of modified T cells may be engineered to release cytokines which have a positive effect on the cytolytic activity of said cells. Such cytokines include, but are not limited to interleukin-7, interleukin-15 and interleukin-21.

Specific Binding TCR/CAR

According to the invention the modified immunoresponsive cells, for example modified T cells, may be modified to express a heterologous TCR or CAR, which binds or specifically binds to tumour cells and/or tissue and/or cancer cells and/or tissue of a subject, patient or cancer patient suffering from a disease condition or cancerous condition optionally which expresses or presents a to a cancer and/or tumour antigen or peptide antigen thereof as herein described. The subject, patient or cancer patient may be subsequently treated with the modified immunoresponsive cell(s) or modified T cell(s) or population thereof according to the invention. Suitable cancer patients for treatment according to the invention with the modified immunoresponsive cells or modified T cells may be identified by a method comprising; obtaining sample of tumour and/or cancer cells from an individual or subject with tumour and/or cancer and; identifying the cancer cells as binding to the TCR or CAR expressed by the modified immunoresponsive cells.

According to the invention the heterologous TCR or CAR binds or specifically binds to a cancer and/or tumour antigen or peptide antigen thereof. According to the invention the heterologous TCR or CAR binds or specifically binds to a cancer and/or tumour antigen or peptide antigen thereof associated with a cancerous condition and/or presented by tumour or cancer cell or tissue.

According to the invention the invention the cancerous condition may be gastroesophageal cancer and/or tumour. Specificity describes the strength of binding between the heterologous TCR or CAR and a specific target cancer and/or tumour antigen or peptide antigen thereof and may be described by a dissociation constant, Kd, the ratio between bound and unbound states for the receptor-ligand system. Additionally, the fewer different cancer and/or tumour antigens or peptide antigen thereof the heterologous TCR or CAR can bind, the greater its binding specificity.

According to the invention the heterologous TCR or CAR may bind to less than 10, 9, 8, 7, 6, 5, 4, 3, 2 different cancer and/or tumour antigens or peptide antigen thereof.

According to the invention the heterologous TCR or CAR may bind, for example to MAGE A4, or an antigenic peptide thereof, for example Human MAGE A4 or MAGE A4 of SEQ ID NO: 1 or an antigenic peptide thereof or to an antigenic peptide comprising or consisting of SEQ ID NO: 2, GVYDGREHTV, with a dissociation constant of between, 0.01 μM and 100 μM, between 0.01 μM and 50 μM, between 0.01 μM and 20 μM, between 0.05 μM and 20 μM or of 0.01, 0.02, 0.03, 0.04, 0.05, 0.06, 0.07, 0.08, 0.09, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1.0 μM, 1.5 μM, 2.0 μM, 2.5 μM, 3.0 μM, 3.5 μM, 4.0 μM, 4.5 μM, 5.0 μM, 5.5 μM, 6.0 μM, 6.5 μM, 7.0 μM, 7.5 μM, 8.0 μM, 8.5 μM, 9.0 μM, 9.5 μM, 10.0 μM; or between 10 μM and 1000 μM, between 10 μM and 500 μM, between 50 μM and 500 μM or of 10, 20 30, 40, 50 60, 70, 80, 90, 100 μM, 150 μM, 200 μM, 250 μM, 300 μM, 350 μM, 400 μM, 450 μM, 500 μM; optionally measured with surface plasmon resonance, optionally at 25° C., optionally between a pH of 6.5 and 6.9 or 7.0 and 7.5. The dissociation constant, K_{D} or k_{off}/kₒₙ may be determined by experimentally measuring the dissociation rate constant, k_{off}, and the association rate constant, kₒₙ. A TCR dissociation constant may be measured using a soluble form of the TCR, wherein the TCR comprises a TCR alpha chain variable domain and a TCR beta chain variable domain. Accordingly, a heterologous TCR or CAR for use in accordance with the invention is capable of binding efficiently and/or with high affinity to HLA displaying GVYDGREHTV, SEQ ID NO: 2 optionally in complex with a peptide presenting molecule for example an HLA, for example with HLA-A*02 or HLA-A*0201, alternatively without presentation in complex with a peptide presenting molecule, for example with a dissociation constant of between 0.01 μM and 100 μM such as 50 μM, 100 μM, 200 μM, 500 μM, preferably between 0.05 μM to 20.0 μM.

According to the present invention the modified immune-responsive cells, for example modified T-cells, may comprise a heterologous TCR or CAR which may bind, specifically bind and/or bind with high affinity to a cancer and/or tumour antigen or peptide antigen thereof optionally associated with a cancerous condition and/or presented by tumour of cancer cell or tissue; optionally wherein the cancer and/or tumour antigen or peptide antigen thereof is recognised by the heterologous TCR or CAR, optionally in complex with a peptide presenting molecule for example an HLA, for example with HLA-A*02 or HLA-A*0201, alternatively without presentation in complex with a peptide presenting molecule for example or HLA (i.e. MAGE-A4 or a peptide antigen thereof or a peptide antigen of MAGE A4 comprising GVYDGREHTV, SEQ ID NO: 2 may be presented independently of a peptide presenting molecule). For example wherein the cancer and/or tumour antigen or peptide antigen thereof is MAGE A4, or an antigenic peptide thereof, for example Human MAGE A4 or MAGE A4 of SEQ ID NO: 1 or an antigenic peptide thereof or an antigenic peptide comprising or consisting of SEQ ID NO: 2, GVYDGREHTV.

According to the present invention the heterologous T cell receptor (TCR) or CAR, and modified immunoresponsive cells comprising the heterologous T cell receptor (TCR) or CAR may have the property of binding to an endogenously expressed tumour cell surface a cancer and/or tumour antigen or peptide antigen thereof optionally wherein the binding is independent of presentation of the cell surface antigen as a complex with an peptide-presenting or antigen-presenting molecule, for example major histocompatibility complex (MHC) or human leukocyte antigen (HLA) or major histocompatibility complex class related protein (MR)1. For example wherein the cancer and/or tumour antigen or peptide antigen thereof is MAGE A4, or an antigenic peptide thereof, for example Human MAGE A4 or MAGE A4 of SEQ ID NO: 1 or an antigenic peptide thereof or an antigenic peptide comprising or consisting of SEQ ID NO: 2, GVYDGREHTV.

According to the present invention the TCR or CAR binding may be specific for one cancer and/or tumour antigen, for example Human MAGE A4 or MAGE A4 of SEQ ID NO: 1 or an antigenic peptide thereof or an antigenic peptide comprising or consisting of SEQ ID NO: 2, GVYDGREHTV, optionally in comparison to a closely related cancer and/or tumour antigen or peptide antigen sequence. The closely related cancer and/or tumour antigen or peptide antigen sequence may be of similar or identical length and/or may have a similar number or identical number of amino acid residues. The closely related peptide antigen sequence may share between 50 or 60 or 70 or 80 to 90% identity, preferably between 80 to 90% identity and/or may differ by 1, 2, 3 or 4 amino acid residues. The closely related peptide sequence may be derived from the polypeptide sequence comprising the sequence or having the sequence GVYDGREHTV, SEQ ID NO: 2.

The binding affinity may be determined by equilibrium methods (e.g. enzyme-linked immunosorbent assay (ELISA) or radioimmunoassay (RIA)), or kinetics (e.g. BIACORE™ analysis). Avidity is the sum total of the strength of binding of two molecules to one another at multiple sites, e.g. taking into account the valency of the interaction. According to the invention the immunoresponsive cells may demonstrate improved affinity and/or avidity to a cancer and/or tumour antigen or peptide antigen thereof, or a cancer and/or tumour antigen or peptide antigen thereof presented by tumour of cancer cell or tissue and recognised by the heterologous TCR or CAR in comparison in comparison to immunoresponsive cells lacking the heterologous TCR or CAR or having an alternative heterologous TCR or CAR.

Selective Binding TCR/CAR

According to the invention, the heterologous TCR or CAR may selectively bind to a cancer and/or tumour antigen or peptide antigen thereof, optionally associated with a cancerous condition and/or presented by tumour of cancer cell or tissue; optionally wherein the cancer and/or tumour antigen or peptide antigen thereof is recognised by the heterologous TCR or CAR, optionally in complex with a peptide presenting molecule for example major histocompatibility complex (MHC) or an HLA, optionally class I or II, for example with HLA-A2, or selected from HLA-A*02, HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02; alternatively without presentation in complex with a peptide presenting molecule or HLA, preferably expressed by a tumour cell or a cancer cell or tissue. Preferably wherein the cancerous condition is gastroesophageal cancer and/or tumour.

Selective binding denotes that the heterologous TCR or CAR binds with greater affinity to one cancer and/or tumour antigen or peptide antigen thereof in comparison to another. Selective binding is denoted by the equilibrium constant for the displacement by one ligand antigen of another ligand antigen in a complex with the heterologous TCR or CAR.

According to the invention the invention the cancerous condition may be gastroesophageal cancer and/or tumour.

Specific/Selective Binding TCR/CAR

According to the present invention the heterologous TCR or CAR binding is selective and/or specific for a cancer and/or tumour antigen or peptide antigen thereof which may be MAGE-A4, or peptide antigen thereof. Preferably the tumour antigen is MAGE-A4 or peptide antigen thereof. Preferably the cancer and/or tumour antigen peptide comprises or has the amino acid sequence GVYDGREHTV, SEQ ID NO: 2. According to the present invention the heterologous TCR or CAR may bind and/or bind specifically and/or bind selectively a peptide presenting molecule for example an HLA presenting or displaying a cancer and/or tumour antigen or peptide antigen thereof, i.e. a peptide fragment of a cancer and/or tumour antigen (pHLA), wherein the HLA corresponds to MHC class I (A, B, and C) which all are the HLA

Class1 or specific alleles thereof or the HLA corresponds to MHC class II (DP, DM, DO, DQ, and DR) or specific alleles thereof, preferably the HLA is class 1, preferably the allele is HLA-A2 or_HLA-A*02 or an HLA-A2+ or_HLA-A*02 positive HLA, preferably HLA-*0201. Preferably the HLA is not HLA-A*02:07P or HLA-A*02 null. Alternatively, the heterologous TCR or CAR may bind and/or bind specifically and/or bind selectively a cancer and/or tumour antigen or peptide antigen thereof, which is not presented or displayed by HLA.

Preferably, the heterologous TCR or CAR is not naturally expressed by the immunoresponsive cells (i.e. the TCR or CAR is exogenous or heterologous). A heterologous TCR may include αβTCR heterodimers. A heterologous TCR or CAR may be a recombinant or synthetic or artificial TCR or CAR i.e. a CAR or TCR that does not exist in nature. For example, a heterologous TCR may be engineered to increase its affinity or avidity for a specific cancer and/or tumour antigen or peptide antigen thereof (i.e. an affinity enhanced TCR or specific peptide enhanced affinity receptor (SPEAR) TCR). The affinity enhanced TCR or (SPEAR) TCR may comprise one or more mutations relative to a naturally occurring TCR, for example, one or more mutations in the hypervariable complementarity determining regions (CDRs) of the variable regions of the TCR α and β chains. These mutations may increase the affinity of the TCR for a peptide fragment of a cancer and/or tumour antigen or peptide antigen thereof or MHCs that display a peptide fragment of a cancer and/or tumour antigen optionally when expressed by tumour and/or cancer cells and/or tissue. Suitable methods of generating affinity enhanced or matured TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901). Preferred affinity enhanced TCRs may bind to tumour or cancer cells expressing the cancer and/or tumour antigen of the MAGE family, for example MAGE A4 or peptide antigen thereof for example peptides thereof comprising or consisting of the sequence GVYDGREHTV, SEQ ID NO: 2.

According to the invention the heterologous TCR may be a MAGE A4 TCR which may comprise the α chain reference amino acid sequence of SEQ ID NO: 5 or a variant thereof and the β chain reference amino acid sequence of SEQ NO: 7 or a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence (for example, with respect to either α chain reference sequence and/or β chain reference sequence). The TCR may be encoded by the α chain reference nucleotide sequence of SEQ ID NO: 6 or a variant thereof and the β chain reference nucleotide sequence of SEQ NO: 8 or a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence (for example, with respect to either α chain reference sequence and/or β chain reference sequence).

According to the present invention the TCR may comprise a TCR alpha chain variable domain and a TCR beta chain variable domain, wherein:

(i) the alpha chain variable domain comprises CDRs having the sequences

VSPFSN (αCDR1), SEQ ID NO:11 or amino acids 48-53 of SEQ ID NO:5,

LTFSEN (αCDR2), SEQ ID NO:12 or amino acids 71-76 of SEQ ID NO:5, and

CVVSGGTDSWGKLQF (αCDR3), SEQ ID NO:13 or amino acids 111-125 of SEQ ID NO:5, and/or

(ii) the beta chain variable domain comprises CDRs having the sequences

KGHDR (βCDR1), SEQ ID NO:14 or amino acids 46-50 of SEQ ID NO:7,

SFDVKD (βCDR2), SEQ ID NO:15 or amino acids 68-73 of SEQ ID NO:7, and

CATSGQGAYEEQFF (βCDR3), SEQ ID NO:16 or amino acids 110-123 of SEQ ID NO:7 or sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto, optionally 100% sequence identity thereto, respectively.

Accordingly, the TCR may comprise a TCR in which the alpha chain variable domain comprises an amino acid sequence that has at least 70%, 75%, 80%, 85%, 90%, 95%, 96%, 97%, 98% or 99% or 100% identity to SEQ ID NO:9 or the sequence of amino acid residues 1-136 of SEQ ID NO:6, and/or the beta chain variable domain comprising an amino acid sequence that has at least 70%, 75%, 80%, 85%, 90%, 95%, 96%, 97%, 98% or 99% or 100% identity to SEQ ID NO:10 or the sequence of amino acid residues 1-133 of SEQ ID NO:7.

The terms “parental TCR” or “progenitor TCR”, is used herein to refer to a TCR comprising the MAGE A4 TCR α chain and MAGE A4 TCR β chain of SEQ ID NOs: 5 and 7 respectively. It is desirable to provide TCRs that are mutated or modified relative to the progenitor TCR that have an equal, equivalent or higher affinity and/or an equal, equivalent or slower off-rate for the peptide-HLA complex than the progenitor TCR. According to the invention the heterologous TCR may have more than one mutation present in the alpha chain variable domain and/or the beta chain variable domain relative to the progenitor TCR and may be denoted, “engineered TCR” or “mutant TCR”. These mutation(s) may improve the binding affinity and/or specificity and/or selectivity and/or avidity for MAGE A4 or peptide antigen thereof. In certain embodiments, there are 1, 2, 3, 4, 5, 6, 7 or 8 mutations in alpha chain variable domain, for example 4 or 8 mutations, and/or 1, 2, 3, 4 or 5 mutations in the beta chain variable domain, for example 5 mutations. In some embodiments, the α chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 9. In some embodiments, the β chain variable domain of the TCR of the invention may comprise an amino acid sequence that has at least 90%, at least 91%, at least 92%, at least 93%, at least 94%, at least 95%, at least 96%, at least 97%, at least 98% or at least 99% identity to the sequence of amino acid residues of SEQ ID NO: 10.

According to the invention the heterologous TCR may comprise a TCR in which, the alpha chain variable domain comprises SEQ ID NO: 9 or the amino acid sequence of amino acid residues 1-136 of SEQ ID NO:5, or an amino acid sequence in which amino acid residues 1-47, 54-70, 77-110 and 126-136 thereof have at least 70%, 75%, 80%, 85%, 90%, 95%, 96%, 97%, 98% or 99% identity to the sequence of amino acid residues 1-47, 54-70, 77-110 and 126-136 respectively of SEQ ID NO:9 and/or in which amino acid residues 48-53, 71-76 and 111-125, CDR 1, CDR 2, CDR 3 respectively, have at least 70%, 75%, 80%, 85%, 90%, 95%, 96%, 97%, 98% or 99% identity to the sequence of amino acid residues 48-53, 71-76 and 111-125, CDR 1, CDR 2, CDR 3, respectively of SEQ ID NO:9.

According to the invention, the TCR may comprise a TCR in which, in the alpha chain variable domain, the sequence of:
(i) amino acid residues 1-47 thereof may have (a) at least 70%, 75%, 80%, 85%, 90% or 95% identity to the sequence of amino acid residues 1-47 of SEQ ID NO:9 or (b) may have one, two or three amino acid residues inserted or deleted relative to residues 1-47 of SEQ ID NO:9,
(ii) amino acid residues 48-53 is VSPFSN, CDR 1, SEQ ID NO:11 or amino acids 48-53 of SEQ ID NO:9,
(iii) amino acid residues 54-70 thereof may have (a) at least 70%, 75%, 80%, 85%, 90% or 95% identity to the sequence of amino acid residues 54-70 of SEQ ID NO: 9 or (b) may have one, two or three amino acid residues inserted or deleted relative to the sequence of amino acid residues 54-70 of SEQ ID NO: 9,
(iv) amino acid residues 71-76 may be LTFSEN, CDR 2, SEQ ID NO:12 or amino acids 71-76 of SEQ ID NO:9,
(v) amino acid residues 77-110 thereof may have at least 70%, 75%, 80%, 85%, 90% or 95% identity to the sequence of amino acid residues 77-110 of SEQ ID NO:9 or may have one, two or three insertions, deletions or substitutions relative to the sequence of amino acid residues 77-110 of SEQ ID NO:9,
(vi) amino acids 111-125 may be CVVSGGTDSWGKLQF, CDR 3, SEQ ID NO:13 or amino acids 111-125 of SEQ ID NO:9,
(vii) amino acid residues 126-136 thereof may have at least 70%, 75%, 80%, 85%, 90% or 95% identity to the sequence of amino acid residues 126-136 of SEQ ID NO: 9 or may have one, two or three insertions, deletions or substitutions relative to the sequence of amino acid residues 126-136 of SEQ ID NO:9.

According to the invention, the TCR may comprise a TCR in which, in the beta chain variable domain comprises the amino acid sequence of SEQ ID NO:10, or an amino acid sequence in which amino acid residues 1-45, 51-67, 74-109, 124-133 thereof have at least 70%, 75%, 80%, 85%, 90% or 95% identity to the sequence of amino acid residues 1-45, 51-67, 74-109, 124-133 respectively of SEQ ID NO:10 and in which amino acid residues 46-50, 68-73 and 110-123 have at least 70%, 75%, 80%, 85%, 90% or 95% identity to the sequence of amino acid residues 46-50, 68-73 and 110-123, CDR 1, CDR 2, CDR 3, respectively of SEQ ID NO:10.

According to the invention, the TCR may comprise a TCR in which, in the beta chain variable domain, the sequence of:
(i) amino acid residues 1-45 thereof may have (a) at least 70%, 75%, 80%, 85%, 90% or 95% identity to the sequence of amino acid residues 1-45 of SEQ ID NO:10 or (b) may have one, two or three amino acid residues inserted or deleted relative to residues 1-45 of SEQ ID NO:10,
(ii) amino acid residues 46-50 is KGHDR, CDR 1, SEQ ID NO:14 or amino acids 46-50 of SEQ ID NO:10,
(iii) amino acid residues 51-67 thereof may have (a) at least 70%, 75%, 80%, 85%, 90% or 95% identity to the sequence of amino acid residues 51-67 of SEQ ID NO:10 or (b) may have one, two or three amino acid residues inserted or deleted relative to the sequence of amino acid residues 51-67 of SEQ ID NO:10,
(iv) amino acid residues 68-73 may be SFDVKD, CDR 2, SEQ ID NO:15 or amino acids 68-73 of SEQ ID NO:10,
(v) amino acid residues 74-109 thereof may have at least 70%, 75%, 80%, 85%, 90% or 95% identity to the sequence of amino acid residues 74-109 of SEQ ID NO:10 or may have one, two or three insertions, deletions or substitutions relative to the sequence of amino acid residues 74-109 of SEQ ID NO:10;
(vi) amino acids 110-123 may be CATSGQGAYEEQFF, CDR 3, SEQ ID NO:16 or amino acids 110-123 of SEQ ID NO:10,
(vii) amino acid residues 124-133 thereof may have at least 70%, 75%, 80%, 85%, 90% or 95% identity to the sequence of amino acid residues 124-133 of SEQ ID NO:10 or may have one, two or three insertions, deletions or substitutions relative to the sequence of amino acid residues 124-133 of SEQ ID NO:10.

According to the invention, the TCR may comprise a TCR which comprises an alpha chain variable domain of SEQ ID NO: 9 and/or a beta chain variable domain of SEQ ID NO: 10. According to the invention, the TCR may comprise a TCR which comprises an alpha chain of SEQ ID NO: 5 and/or a beta chain of SEQ ID NO: 7.

Amino acid and nucleotide sequence identity is generally defined with reference to the algorithm GAP (GCG Wisconsin Package™, Accelrys, San Diego Calif.). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), generally employing default parameters.

Particular amino acid sequence variants may differ from a reference sequence by insertion, addition, substitution or deletion of 1 amino acid, 2, 3, 4, 5-10, 10-20 or 20-30 amino acids.

In some embodiments, a variant sequence may comprise the reference sequence with 1, 2, 3, 4, 5, 6, 7, 8, 9, 10 or more residues inserted, deleted or substituted. For example, up to 15, up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:

alanine and glycine;

glutamic acid, aspartic acid, glutamine, and asparagine

arginine and lysine;

asparagine, glutamine, glutamic acid and aspartic acid

isoleucine, leucine and valine;

phenylalanine, tyrosine and tryptophan

serine, threonine, and cysteine.

The CD8α co-receptor

According to the present invention, the modified immunoresponsive cells expressing or presenting a heterologous TCR or CAR may further express or present a heterologous co-receptor (e.g., the cell is transduced with or engineered to comprise a nucleic acid sequence encoding a co-receptor, for example by gene knock in). The heterologous co-receptor may be a CD8 co-receptor. The CD8 co-receptor may comprise a dimer or pair of CD8 chains which comprises a CD8-α and CD8-β chain or a CD8-α and CD8-α chain. Preferably, the CD8 co-receptor is a CD8αα co-receptor comprising a CD8-α and CD8-α chain. A CD8α co-receptor may comprise the amino acid sequence of at least 80% identity to SEQ ID NO: 3, SEQ ID NO: 3 or a variant thereof. The CD8a co-receptor may be a homodimer. Preferably the modified immunoresponsive cells expressing or presenting a heterologous TCR or CAR further express or present a heterologous co-receptor (e.g., the cell is transduced with or engineered to comprise a nucleic acid sequence encoding a co-receptor, for example by gene knock in) which is a CD8 co-receptor as defined herein.

The CD8 co-receptor binds to class 1 MHCs and potentiates TCR signalling. According to the invention the CD8 co-receptor may comprise the reference amino acid sequence of SEQ ID NO: 3 or may be a variant thereof. A variant may have an amino acid sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference amino acid sequence SEQ ID NO: 3. The CD8 co-receptor may be encoded by the reference nucleotide sequence of SEQ ID NO: 4 or may be a variant thereof. A variant may have a nucleotide sequence having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity to the reference nucleotide sequence SEQ ID NO: 4.

According to the invention the heterologous CD8 co-receptor may comprise a CD8 co-receptor in which, in the Ig like V-type domain comprises CDRs having the sequence;

(i) VLLSNPTSG, CDR1, SEQ ID NO: 17, or amino acids 45-53 of SEQ ID NO: 3,

(ii) YLSQNKPK, CDR2, SEQ ID NO: 18 or amino acids 72-79 of SEQ ID NO: 3,

(iii) LSNSIM, CDR3, SEQ ID NO: 19 or amino acids 80-117 of SEQ ID NO: 3,

or sequences having at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or at least 99% sequence identity thereto.

According to the invention the heterologous CD8 co-receptor may comprise a CD8 co-receptor which comprises or in which, in the Ig like V-type domain comprises, residues 22-135 of the amino acid sequence of SEQ ID No:3, or an amino acid sequence in which amino acid residues 22-44, 54-71, 80-117, 124-135 thereof have at least 70%, 75%, 80%, 85%, 90% or 95% identity to the sequence of amino acid residues 22-44, 54-71, 80-117, 124-135, CDR 1, CDR 2, CDR 3, respectively of SEQ ID No:3 and in which amino acid residues 45-53, 72-79 and 118-123 have at least 70%, 75%, 80%, 85%, 90% or 95% identity to the sequence of amino acid residues 45-53, 72-79 and 118-123 respectively of SEQ ID No:3.

According to the invention the CD8 co-receptor may comprise a CD8 co-receptor in which, or in which in the Ig like V-type domain, the sequence of:
(i) amino acid residues 22-44 thereof may have (a) at least 70%, 75%, 80%, 85%, 90% or 95% identity to the sequence of amino acid residues 22-44 of SEQ ID NO:3 or (b) may have one, two or three amino acid residues inserted or deleted relative to residues 22-44 of SEQ ID NO:3,
(ii) amino acid residues 45-53 is VLLSNPTSG, SEQ ID NO:17, CDR1, or amino acids 45-53 of SEQ ID NO:3,
(iii) amino acid residues 54-71 thereof may have (a) at least 70%, 75%, 80%, 85%, 90% or 95% identity to the sequence of amino acid residues 54-71 of SEQ ID NO:3 or (b) may have one, two or three amino acid residues inserted or deleted relative to the sequence of amino acid residues 54-71 of SEQ ID NO:3,
(iv) amino acid residues 72-79 may be YLSQNKPK, CDR2, SEQ ID NO:18 or amino acids 72-79 of SEQ ID NO:3,
(v) amino acid residues 80-117 thereof may have at least 70%, 75%, 80%, 85%, 90% or 95% identity to the sequence of amino acid residues 80-117 of SEQ ID NO:3 or may have one, two or three insertions, deletions or substitutions relative to the sequence of amino acid residues 80-117 of SEQ ID NO:3;
(vi) amino acids 118-123 may be LSNSIM, CDR3, SEQ ID NO:19 or amino acids 80-117 of SEQ ID NO:3,
(vii) amino acid residues 124-135 thereof may have at least 70%, 75%, 80%, 85%, 90% or 95% identity to the sequence of amino acid residues 124-135 of SEQ ID NO:3 or may have one, two or three insertions, deletions or substitutions relative to the sequence of amino acid residues 124-135 of SEQ ID NO:3.

The modified immunoresponsive cells that express heterologous CD8 co-receptor may demonstrate improved affinity and/or avidity and/or improved T-cell activation, as determinable by the assays disclosed herein, towards or on stimulation by antigenic peptide, tumour or cancer antigen optionally when presented on HLA relative to modified immunoresponsive cells that do not express heterologous CD8 co-receptor. The heterologous CD8 of modified immunoresponsive cells may interact or bind specifically to an MHC, the MHC may be class I or class II, preferably class I major histocompatibility complex (MHC), HLA-I molecule or with the MHC class I HLA-A/B2M dimer, preferably the CD8-α interacts with the α₃ portion of the Class I MHC (between residues 223 and 229), preferably via the IgV-like domain of CD8. Accordingly the heterologous CD8 improves TCR binding of the immunoresponsive cells to the HLA and/or antigenic peptide bound or presented by HLA pMHCI or pHLA, optionally on the surface of antigen presenting cell, dendritic cell and/or tumour or cancer cell, tumour or cancer tissue compared to immunoresponsive cells lacking the heterologous CD8. Accordingly the heterologous CD8 can improve or increase the off-rate (k_{off}) of the cell (TCR)/peptide-major histocompatibility complex class I (pMHCI) interaction of the immunoresponsive cells, and hence its half-life, optionally on the surface of antigen presenting cell, dendritic cell and/or tumour or cancer cell, or tumour or cancer tissue compared to the cells lacking the heterologous CD8, and thereby may also provide improved ligation affinity and/or avidity. The heterologous CD8 can improve organizing the TCR on the immunoresponsive cell surface to enable cooperativity in pHLA binding and may provide improved therapeutic avidity. Accordingly, the heterologous CD8 co-receptor modified immunoresponsive cells may bind or interact with LCK (lymphocyte-specific protein tyrosine kinase) in a zinc-dependent manner leading to activation of transcription factors like NFAT, NE-κB, and AP-1.

According to the invention the modified immunoresponsive cells may have an improved or increased expression of CD40L, cytokine production, cytotoxic activity, induction of dendritic cell maturation or induction of dendritic cell cytokine production, optionally in response to cancer and/or tumour antigen or peptide antigen thereof optionally as presented by tumour of cancer cell or tissue, in comparison to immunoresponsive cells lacking the heterologous CD8 co-receptor.

Therapy

Gastroesophageal cancer and/or tumour encompasses cancer and/or tumour of the esophagus, gastro-esophageal junction or stomach, i.e. gastric cancer.

According to the present invention the cancer and/or tumour may be gastroesophageal cancer and/or tumour, for example esophageal cancer, carcinoma or tumour, which may be primary, secondary, recurrent, metastatic or advanced. Preferably the esophageal cancer, carcinoma or tumour may be selected from any one of; esophageal squamous-cell carcinoma (ESCC), esophageal adenocarcinoma (EAC), or esophagogastric junction cancer, carcinoma adenocarcinoma or tumour (EGJ), optionally associated with gastroesophageal reflux disease GERD or Barrett's esophagus.

Accordingly the cancer and/or tumour may be gastroesophageal cancer and/or tumour, for example, esophageal squamous-cell carcinoma or non-epithelial tumors, such as leiomyosarcoma, malignant melanoma, rhabdomyosarcoma or lymphoma.

Accordingly the cancer and/or tumour may be gastroesophageal cancer and/or tumour, for example, upper, middle or lower junctional esophageal cancer, carcinoma or tumour.

Accordingly the and/or tumour may be gastroesophageal cancer and/or tumour, for example, esophageal cancer, carcinoma or tumour which involves the lymph nodes or which is metastasised for example to lymph nodes, liver, lungs, bone or head and neck cancers.

Accordingly the may be gastroesophageal cancer and/or tumour, for example and/or esophageal cancer and/or tumour, carcinoma or tumour may express a MAGE protein, peptide, antigen or peptide antigen thereof, optionally MAGE-A4 protein, peptide, antigen or peptide antigen thereof as herein described. According to the invention the cancer and/or tumour may be recurrent or metastatic HNSCC, optionally with disease progression on after platinum containing chemotherapy, optionally expressing MAGE-A4 protein, peptide, antigen or peptide antigen thereof as described herein (for example a peptide antigen of MAGE A4 comprising GVYDGREHTV, SEQ ID NO: 2).

Where esophagogastric junction cancer is lower junctional esophageal cancer, it has a high likelihood of spreading to the stomach. Accordingly, according to the present invention the cancer may be gastroesophageal cancer and/or tumour, for example stomach or gastric cancer, carcinoma or tumour, which may be primary, secondary, recurrent, metastatic or advanced.

Preferably the stomach or gastric cancer, carcinoma or tumour may be selected from any one of; gastric carcinoma, gastric adenocarcinoma, gastric lymphoma, extranodal marginal zone B-cell lymphomas (MALT type), diffuse large B-cell lymphomas, mesenchymal tumors of the stomach, hereditary diffuse gastric cancer.

Accordingly the cancer may be gastroesophageal cancer and/or tumour, for example, stomach or gastric cancer, carcinoma or tumour which involves the lymph nodes or which is metastasised for example to the liver, lungs, bones, lining of the abdomen and lymph nodes.

Accordingly the stomach or gastric cancer, carcinoma or tumour may express a MAGE protein, peptide, antigen or peptide antigen thereof, optionally MAGE-A4 protein, peptide, antigen or peptide antigen thereof as herein described. According to the invention the cancer may be recurrent or metastatic HNSCC, optionally with disease progression on after platinum containing chemotherapy, optionally expressing MAGE-A4 protein, peptide, antigen or peptide antigen thereof as described herein (for example a peptide antigen of MAGE A4 comprising GVYDGREHTV, SEQ ID NO: 2).

Standard of Care

Gastroesophageal cancer and/or tumour encompasses cancer and/or tumour of the esophagus, gastro-esophageal junction or stomach, i.e. gastric cancer.

The standard of care for Gastroesophageal cancer and/or tumour, optionally including esophagogastric Junction (EGJ) cancer and/or tumour, can be a systemic platinum-based chemotherapy and may be selected from cisplatin or carboplatin chemotherapy treatment, alternatively any of combined cisplatin, fluorouracil, and leucovorin or cisplatin and etoposide or carboplatin and paclitaxel, or cisplatin and capecitabine, alternatively a platinum fluoropyrimidine doublet and optionally anthracycline. Alternatively the standard of care can be selected from treatment with any one of ECF (epirubicin, cisplatin, 5-FU), ECX (epirubicin, cisplatin, capecitabine), EOF (epirubicin, oxaliplatin, 5-FU), and EOX (epirubicin, oxaliplatin, capecitabine).

Alternatively the standard of care can be selected from treatment with any one of lapatinib, FLOT/FOLFIRI chemotherapy 5-fluorouracil, epirubicin, etoposide, docetaxel, oxaliplatin, capecitabine or irinotecan, ifosfamide, mytomycin C, vindesine, vinblastine, etoposide, gemcitabine, or a taxane such as paclitaxel (Taxol) or docetaxel. Alternatively the standard of care can be selected from a targeted therapy for example with a human epidermal growth factor receptor 2 (HER2) inhibitor, such as any of trastuzumab, lapatinib or pertuzamab or a vascular endothelial growth factor (VEGF) inhibitor such as for example ramucirumab or bevacizumab or an EGFR inhibitor antibody, such as panitumumab or cetuximab or a human hepatocyte growth factor HGF and/or Met inhibitor, such as onartuzumab or rilotumumab or a PD1, PD-L1 inhibitor such as nivolumab, pembrolizumab, avelumab, atezolizumab, or cetuximab or cetuximab in combination with any one of fluorouracil, methotrexate, cisplatin, carboplatin or a taxane such as paclitaxel (Taxol) or docetaxel.

Accordingly the present invention and the methods, treatment and uses of the present invention provides a reduction in MAGE-A4 expression or concentration in a subject in comparison to placebo treatment or in comparison to without treatment or compared to pre-treatment, or in comparison to treatment comprising a standard of care.

Disease Biomarkers

The present invention and the methods, treatment and uses and/or kits of the present invention provides a treatment, prevention or delay in the progression of Gastroesophageal cancer and/or tumour in a subject as determined by subject disease biomarker changes in expression or concentration compared to the pre-treatment disease biomarker expression or concentration or in comparison placebo treatment or to without treatment or in comparison to treatment comprising a standard of care.

Changes in disease biomarker levels from Baseline (pre-treatment) are correlated with response to treatment and correspond to treatment efficacy and success of cancer and/or tumour treatment.

Disease biomarkers of Gastroesophageal cancer can include any one or more of; carbohydrate antigen (CA) 72-4, CA19-9 glycolipid antigen, alpha-fetoprotein, carbohydrate antigen (CA)12-5, SLE, BCA-225, hCG, pepsinogen I/II, carcinoembryonic antigen (CEA), HER2 proto-oncogene, CD44, or CA19-9, which are frequently used biomarkers in clinical practice for gastroesophageal cancer and/or tumour

Disease biomarkers of Gastroesophageal cancer can also include any one of expression levels or plasma levels of VEGF-A or Angiopoietin-2 (Ang-2), over-expression or gene amplification of any of EGFR, mesenchymal-epithelial transition factor receptor (MET), hepatocellular growth factor (HGF), FGFR1, FGFR2, FGFR3 and FGFR4 (fibroblast growth factor receptors), gene amplification of FGFR induces receptor overexpression, chromosomal translocation, and point mutation or enhanced kinase activity, expression levels of PD-1, PD-L1, elevated levels of programmed death ligands 1 and 2 (PD-L1/2), gene mutations in known cancer-related genes in gastroesophageal cancer and/or tumour, such as TP53, PTEN, ARID1A, APC, CTNNB1, CDH1, PI3KCA and KMT2C.

Disease biomarkers of Gastroesophageal cancer and/or tumour may also be selected from any one or more of; gene mutations, deletions or copy number alterations in any of the following genes (tumour series) TP53, ELMO1, DOCK2, CDKN2A, ARID1A, SMAD4, PIK3CA, KRAS, HER2, EGFR, CND1, MET.

EBV-positive subtype gastroesophageal cancer and/or tumour biomarkers can include any of; CXXC4, TIMP2 and PLXND1. COL9A2, EYA1 and ZNF365 are highly methylated in EBV-positive, or somatic mutation of BMP (bone morphogenetic protein), or amplification or mutation of any of JAK2, MET, ERBB2, PIK3CA or mutations in ARID1A or BCO.

Disease biomarkers of Gastroesophageal cancer and/or tumour may also be selected from any one or more of; CXC chemokine receptor 2 (CXCR2) expression or mRNA expression, CC chemokine receptor 4 (CCR4) expression or mRNA expression, CC chemokine receptor 7 (CCR7) expression or mRNA expression, detected loss of heterozygosity in tumour cell derived DNA, the presence or level of hypermethylation of cytosine-phosphate-guanine (CpG)-rich promoter regions, metalloproteinase expression, e.g. MMP-1 or the gelatinases MMP-2 or MMP-9 or the stromelysins, MMP-3 and MMP-10, Interleukin IL-6 and IL-8 levels or expression, expression of MAGE-A1, 2, 3, 4, 5, 6, cytokeratin (e.g. CKs 6, 16 or 17) or actin or myosin concentration or expression levels, overexpression of eukaryotic translation factor 4E (eIF4E), mutation levels in DNA repair genes e.g. of the nucleotide excision repair (NER) group.

Disease biomarkers of gastroesophageal cancer and/or tumour, may be further selected from any one or more of; presence or level of anaplastic lymphoma kinase (ALK) translocations, presence or level of epidermal growth factor receptor (EGFR) mutations, presence or level of Kirsten rat sarcoma viral oncogene homolog (KRAS) mutations, human epidermal growth factor receptor-2 (HER2/neu) expression, presence or level of B-Raf proto-oncogene serine/threonine kinase (BRAF) mutations, presence or level of C-KIT proto-oncogene mutations, expression of MAGE-A1, 2, 3, 4, 5, 6, presence or level of Janus kinase 2 (JAK2) mutations.

Further disease biomarkers of gastroesophageal cancer and/or tumour, may include any one of EML4-ALK tyrosine kinase fusion, epigenetic changes such as alteration of DNA methylation, histone tail modification, or microRNA regulation causing inactivation of tumour suppression, mutations and amplification of c-MET, NKX2-1, LKB1, PIK3CA, and BRAF.

Disease biomarkers gastroesophageal cancer and/or tumour, can also include circulating tumour cells (CTCs), cell-free DNA, micro RNA, cell-free RNA and cell-derived vesicles, such as exosomes which may circulate in a biological sample as described herein. CTCs are disseminated tumour cells which are circulating in the bloodstream their presence is clinically related with the cancer or progressive or metastatic disease.

According to the invention disease biomarkers may be measured in a biological sample of a subject for example as described herein.

Biological Sample

A biological sample can be any subject or patient body fluid that may contain a disease biomarker or cell or genetic material from a cancer or tumour (e.g. gastroesophageal cancer and/or tumour i.e. cancer and/or tumour of the esophagus, gastro-esophageal junction or stomach, i.e. gastric cancer), for instance blood, serum, plasma, urine, tissue, cells, cell cultures, saliva, sputum, cerebrospinal fluid, lavage or fluid from lung, nasal, bronchus, bronchoalveolar, esophagogastric or gastrointestinal tract, gastric or esophagastric wash or gastric juice, peripheral blood samples from patients or subjects with cancer containing circulating tumour cells (CTCs), cell-free DNA, micro RNA, cell-free RNA and cell-derived vesicles, such as exosomes. CTCs are disseminated tumour cells as single cells or, less commonly, as cell clusters, derived from either primary tumours or metastases which are circulating in the bloodstream. The existence of CTCs like other disease biomarkers are clinically related with tumour and/or cancer, progressive or metastatic disease. For example, in gastroesophageal cancer and/or tumour, disease biomarkers or biomarkers or MAGE-A4 or antibodies thereto may be detected in a biological sample for example a body fluid for example fluid from esophagogastric or gastrointestinal tract, gastric or esophagastric wash or gastric juice, as biomarkers of MAGE-A4 expressing cancer and/or tumour and/or tissue.

Therapeutic Effect

Serum Cytokine and Soluble Factor Analysis and T-Cell Infiltration of Tumour

The present invention and the methods, treatment and uses of the present invention provides an increase in serum cytokine and/or interferon level or concentration in a subject compared to the pre-treatment serum cytokine and/or interferon level or concentration or in comparison to placebo treatment or without treatment or treatment comprising a standard of care.

Accordingly the invention and the methods, treatment and uses of the present invention provides an improved or enhanced cancer and/or tumour immunogenicity, for example as measured by the ability to provoke an immune response in response to cancer and/or tumour or cancer and/or tumour antigen, for example enhanced by at least 10%, alternatively 20%, 30%, 40%, 50%, 60%, 70%, 80%, 90%, 100%, 120%, 150%, 200% or more relative to such levels before the treatment or intervention or compared to placebo, or relative to without treatment or relative to treatment comprising a standard of care, for example as judged by increased secretion of cytokines and/or interferon, increased T-cell proliferation, increased antigen responsiveness, target cell killing, T-cell activation, CD28 signalling, T-cell infiltration of tumour, ability to recognise and bind to dendritic cell presented antigen.

The efficacy of immunotherapy of cancer is conditioned by the infiltration of tumours by activated tumour-specific T-cells. The activity of these T-cells will in turn be affected by the presence in the tumour of an immunosuppressive environment (e.g. regulatory T-cells). Therefore, the direct evaluation of the “immune landscape” inside the tumour is of great value for monitoring efficacy of the T-cell immunotherapy and may be quantitated by tumour biopsies to evaluate the immune status of the tumour before and after T-cell infusion. Accordingly the invention provides an improved T-cell infiltration of tumour and/or reduction in T-cell repressive factors as determined for example by a reduction in level of T-regs, Myeloid derived suppressor cells (MDSCs), PD-L1 protein expression, serum cytokine levels selected from CCL3, IL8, IL1β, CXCL10, or sIL2Rα or levels of inhibitory receptors, selected from PD-1, CTLA-4, TIM-3, LAG-3, BTLA or TIGIT compared to pre-treatment or without treatment or in comparison to treatment comprising a standard of care. Alternatively as determined from an increase in level of interferon-γ, interleukin-6, interleukin-10, cytokine production, such as IL-2, TNF-α, IFN-γ and granzyme B or innate immune cells such as NK cells, adaptive immune cells (CD4⁺ and CD8⁺) or improved proliferation in T-cells for example as judged by Ki67 expression level, compared to pre-treatment or without treatment or in comparison to treatment comprising a standard of care as herein before described.

Tumour Size and Tumour Burden

The present invention and the methods, treatment and uses of the present invention provides an improved or enhanced level or response of reducing tumour growth or tumour growth rate or maintaining tumour size after cessation of treatment or of tumour number or tumour burden, in comparison to prior to treatment or treatment with placebo or without treatment or treatment comprising a standard of care, for example, as determined by the measurement of tumour size or tumour number, preferably improved or enhanced by at least 10%, alternatively 20%, 30%, 40%, 50%, 60%, 70%, 80%, 90%, 100%, 120%, 150%, 200% or more relative to prior to treatment, treatment with placebo, or without treatment or treatment comprising a standard of care. Preferably an improved or enhanced level or response may be a sustained improved or enhanced level or response and/or may have a duration at least the same as the treatment duration, at least 1.5, 2.0, 2.5, or 3.0 or more times the length of the treatment duration. Such improved or enhanced level or response may be judged from RECIST 1.1 measurements [E. A. Eisenhauer., et.al., EUROPEAN JOURNAL OF CANCER 45 (2009) 228-247] or by tumour biopsy or liquid biopsy (plasma from peripheral blood) to determine tumour related circulating-free DNA (cfDNA) or exosomes (source of stable mRNA). Exosomes (produced by all cells, including tumour cells and immune cells) and cfDNA (produced by dying tumour cells) may be used to monitor both the tumour burden and the immune response. The analysis of exosomes and cfDNA may allow: (a) estimation and genetic profiling of the global tumour burden (including expression of MAGE-A4 mRNA or mutational profiling) from exosomes and cfDNA, (b) Systemic assessment of the immune response (gene expression by cytotoxic and regulatory immune cells) from exosomes.

According to the foregoing, the standard of care treatment may be as herein above described for gastroesophageal cancer and/or tumour.

MAGE-A4 TCR+ Cell Persistence

The present invention and the methods, treatment and uses of the present invention provides improved therapeutic effect and improved treatment, prevention or delaying in the progression of gastroesophageal cancer and/or tumour in a subject, in comparison to prior to treatment, treatment with placebo or without treatment or treatment comprising a standard of care, for example, as determined by the measurement of the persistence of infused engineered and modified immunoresponsive cells expressing or presenting a heterologous T-cell receptor (TCR) as herein described. Persistence of the infused engineered and modified immunoresponsive cells is correlated with therapeutic effect and is also a long-term safety measure. Cell persistence can be determined by qPCR or flow cytometry (FCM). For example the quantitation of MAGE-A4 or MAGE-A4+CD8 TCR+ cells by PCR of transgene from DNA extracted from frozen subject PBMC may be used as a measure, likewise the quantitation of MAGE-A4 or MAGE-A4+CD8 TCR expressing cells by FCM from frozen subject PBMC. T cell phenotype and activity may be determined by a range of assays, for example:
Phenotype analysis for determination of T-cell lineages in cell product and in the subject blood pre and post infusion.
Quantitation of the senescence and activation status of T-cells from subject PBMC
Quantitation of soluble factors reflecting in vivo function of infused T cells, for example MAGE-A4 or MAGE-A4+CD8 TCR+ T-cells.
Ex-vivo activity of transduced cells of subjects at different time points to assess potential functionality of those cells before and/or during the timeframe of treatment.

T-Cell Function

The present invention and the methods, treatment and uses of the present invention provides an enhancement of T-cell function compared to pre-treatment, treatment with placebo or in comparison to without treatment or in comparison to treatment comprising a standard of care. Preferably the T-cell function is enhanced by at least 10%, alternatively 20%, 30%, 40%, 50%, 60%, 70%, 80%, 90%, 100%, 120%, 150%, 200% or more, for example as judged by increased secretion of y-interferon from CD8⁺ T-cells, increased T-cell proliferation, increased internal signalling, increased antigen responsiveness, increased secretion of cytokines and/or interferon, increased target cell killing, increased T-cell activation, increased CD28 signalling, increased T-cell ability to infiltrate tumour, increased ability to recognise and bind to dendritic cell presented antigen.

According to the present invention and the methods and uses of the present invention, tumour immunity or evasion of immune recognition by the tumour may be attenuated resulting in improved tumour recognition and attack by the immune system and thereby treating tumour immunity for example as measured by tumour binding, tumour shrinkage and tumour clearance. Accordingly, the present invention provides treatment of tumour immunity and/or provides treatment of tumour immunity which is enhanced by at least 10%, alternatively 20%, 30%, 40%, 50%, 60%, 70%, 80%, 90%, 100%, 120%, 150%, 200% or more compared to pre-treatment, treatment by placebo or in comparison to without treatment or in comparison to treatment comprising a standard of care, for example as measured by tumour binding, tumour shrinkage or tumour clearance.

In the context of T-cell activity the term “dysfunction” refers to a state of reduced immune responsiveness to antigenic stimulation and includes T-cell exhaustion and/or anergy whereby the T-cell may recognise and bind antigen, e.g. cancer and/or tumour antigen or peptide antigen thereof, but shows reduced effectiveness in progressing immune response or combating cancer progress and/or tumour growth. Dysfunctional T-cells demonstrate impaired capacity to translate antigen recognition into down-stream T-cell effector functions, such as proliferation, cytokine and interferon production or target cell killing and/or appear refractory or unresponsive to antigen recognition as is characteristic of T-cell dysfunctional disorder. “T-cell dysfunctional disorder” may be associated with or detected as inappropriate increased T-cell signalling through PD-1; T-cells having decreased ability to proliferate and/or produce cytokines and/or cytolytic activity; T-cell anergy; tumour immunity.

“T-cell exhaustion” comprises a state of T cell dysfunction due to sustained TCR signalling as part of the response to cancer and prevents optimal response to tumours. Exhaustion can find effect through either the cell intrinsic negative regulatory (costimulatory) pathways (for example PD-1, PD-1 axis, B7-H3, B7-H4) or through the cell extrinsic negative regulatory pathways (immunoregulatory cytokines). T-cell exhaustion is characterised by poor effector function, sustained expression of inhibitory receptors and an altered activity of transcription distinct from that of functional effector or memory T-cells. T-cell anergy occurs through deficient signalling through the T-cell receptor and a resulting state of unresponsiveness to antigen stimulation often even in the context of co-stimulation, consequently such T-cells do not undergo clonal expansion and/or acquire effector functions.

Treatment and Administration

According to the invention the modified immunoresponsive cells may be administered continuously or intermittently, optionally as a single dose or as more than one dose.

Accordingly the modified immunoresponsive cells may be administered as a single dose or as more than one dose (multiple doses). The modified immunoresponsive cells may be administered at a dose of between about 500 million to any one of about 1 billion cells, about 2 billion cells, about 3 billion cells, about 4 billion cells, about 5 billion cells, about 6 billion cells, about 7 billion cells, about 8 billion cells, about 9 billion cells, about 10 billion cells, about 11 billion cells, about 12 billion cells, about 13 billion cells, about 14 billion cells, about 15 billion cells, about 16 billion cells, about 17 billion cells, about 18 billion cells, about 19 billion cells, about 20 billion cells, or about 21 billion cells. The modified immunoresponsive cells may be administered at a dose of between about 100 million to about 200 million cells, about 300 million to about 400 million cells, about 500 million to about 600 million cells, about 700 million to about 800 million cells, or about 900 million to about 1 billion cells, optionally about 500 million to about 1 billion cells, about 2 billion to about 5 billion cells or about 6 billion to about 10 billion cells.

According to the invention the modified immunoresponsive cells may be administered, intravenously, intramuscularly, subcutaneously, topically, orally, transdermally, intraperitoneally, intraorbitally, by implantation, by inhalation, intrathecally, intraventricularly, or intranasally or by intravenous infusion. Preferably, the modified immunoresponsive cells may be administered intravenously or by intravenous infusion.

According to the invention modified immunoresponsive cells can be administered as

(a) a single dose in each of one or more dosing cycles,

(b) one or more doses in each of one or more dosing cycles,

(c) a single dose on the first day of each of one or more dosing cycles,

(d) one or more doses in each of one or more dosing cycles comprising a dose on the first day of each of the one or more dosing cycles,

(e) one or more doses in each of one or more dosing cycles, at least one dose being on the first day of each cycle,

(f) a single dose.

According to the invention modified immunoresponsive cells can be administered in a dosing cycle wherein the dosing cycle can be any of 12, 13, 14, 15, 16, 17, 18, 19, 20, 21, 22, 23, 24, 25, 26, 27, 28 weeks or any of 1 month, 2 months, 3 months, 4 months, 5 months, 6 months, 7 months, 8 months, 9 months, 10 months, 11 months, or 12 months, (e.g. since the last previous dose). Accordingly the dosing cycle can be any of 10 to 12 weeks, 11 to 13 weeks, 14 to 17 weeks, 14 to 17 weeks, 18 to 21 weeks, 22 to 24 weeks, 24 to 27 weeks, 28 to 30 weeks, 3 months, 4 months, 5 months, 6 months, (e.g. since the last previous dose).

According to the invention modified immunoresponsive cells can be administered in a dosing cycle wherein the dosing cycle can be on, or commence on or re-commence on:

(a) disease progression following a previous administration of modified immunoresponsive cells, and/or

(b) 12 weeks or more following the previous administration of modified immunoresponsive cells, and wherein

(c) the tumour and/or cancer expresses MAGE-A4 and/or peptide antigen thereof and/or

(d) MAGE-A4 and/or peptide antigen thereof is detected in a subject biological sample and/or is above the normal range.

According to the invention modified immunoresponsive cells can be administered in a dosing cycle wherein the dosing cycle can be on, or commence on or re-commence on:
(a) confirmed response or complete response or partial response following a previous administration of modified immunoresponsive cells, or (b) stable disease for a period of greater than or equal to 2, 3, or 4 months followed by disease progression following the previous administration of modified immunoresponsive cells, and/or
(c) greater than or equal to 12 weeks following the previous administration of modified immunoresponsive cells, and wherein

(c) the tumour and/or cancer expresses MAGE-A4 and/or peptide antigen thereof and/or

(d) MAGE-A4 and/or peptide antigen thereof is detected in a subject biological sample and/or is above the normal range.

The tumour and/or cancer may express MAGE-A4 and/or peptide antigen thereof at a level greater than or equal to an intensity of 1+ in and/or antigen expression frequency by immunohistochemistry of, greater than or equal to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% preferably greater than or equal to 30 or 32% of tumour and/or cancer cells as determined by immunohistochemistry. The subject biological sample MAGE-A4 and/or peptide antigen thereof which is above the normal range may be greater than or equal to 10, 20, 30, 40, 50, 60, 70, 80, 90, 100, 150, 200, 250, 300, 350, 400, 450 or 500 ng/mL, preferably greater than or equal to 50 or 100 ng/mL.

According to the invention the dose may be a fixed dose or a variable dose. For example where more than one dose is administered, i.e. multiple dose, the dose may be fixed or may be variable, for example where more than one dose is administered the dose may be escalated or increased, for example in each dosing cycle, i.e. may be of increasing level of dose, for example in progression, for example 100 million to 500 million to 1 billion to 5 billion to 10 billion cells.

According to the invention the modified immunoresponsive cells are preferably administered as a single dose of between about 5 billion and about 10 billion cells.

According to the invention the modified immunoresponsive cells can administered for a specified period, meaning that the modified immunoresponsive cells dosing cycles can administered for a specified period. The specified period may be any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 months, preferably 24 months.

According to the invention the method may comprise the steps wherein

(a) the the modified immunoresponsive cells are administered as a single dose,

(b) the status of disease is determined at a period after the modified immunoresponsive cells administration and compared to the status prior to the modified immunoresponsive cells administration, wherein if progressive disease is determined then,

(c) modified immunoresponsive cells are administered as a single dose, optionally wherein the tumour and/or cancer expresses MAGE-A4 and/or peptide antigen thereof and/or MAGE-A4 and/or peptide antigen thereof is detected in a subject biological sample and/or is above the normal range. Preferably the period is greater than or equal to any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 weeks, preferably greater than or equal to 12 weeks

According to the invention the method may comprise the steps wherein

(a) the modified immunoresponsive cells are administered as a single dose,

(b) the status of disease is determined at a first and a later second period after the modified immunoresponsive cells administration and compared to the status prior to the modified immunoresponsive cells administration, wherein if stable disease is determined after the first period and progressive disease is determined after the second period then,

(c) modified immunoresponsive cells are administered administered as a single dose, optionally wherein the tumour and/or cancer expresses MAGE-A4 and/or peptide antigen thereof and/or MAGE-A4 and/or peptide antigen thereof is detected in a subject biological sample and/or is above the normal range. Preferably the first period is greater than or equal to any one of 1, 2, 3, 4, 5, 6, 7, 8, months, preferably greater than or equal to 4 months.

Preferably the second period is greater than or equal to any one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 months after the first period, preferably greater than or equal to 4 months.

According to the invention a “complete response” (CR) is determined where all target lesions or tumours have been assessed or measured as having disappeared. “Partial response” (PR) is determined when there is a measurement of an at least a 30% decrease in the sum of the longest diameters (SLD) of target lesions or tumours, for example as referenced to the control or pre-treatment comparator. “Progressive disease” (PD) is determined when there is a measurement of at least a 20% increase in the sum of the longest diameters (SLD) of target lesions or tumours, for example as referenced to the control or pre-treatment comparator, since the treatment started or the presence of one or more new lesions. “Stable disease” (SD) is determined where it is determined that there is neither sufficient reduction or decrease in the sum of the longest diameters (SLD) of target lesions or tumours to qualify for PR, nor sufficient increase to qualify for PD, taking as reference the smallest SLD since the treatment started.

According to the present invention the subject prior to treatment can comprise tumour and/or cancer cell MAGE-A4 and/or peptide antigen thereof expression of greater than or equal to an intensity of 1+ in and/or antigen expression frequency by immunohistochemistry of, greater than or equal to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% preferably greater than or equal to 30 or 32% of tumour and/or cancer cells as determined by immunohistochemistry and non-cancerous MAGE-A4 and/or peptide antigen thereof expression is less than or equal to 1, 2, 3, 5, 6, 7, 8, 9, 10% preferably less than or equal to 1% or 5% of cells for non-cancerous or non-tumour tissue at any intensity by immunohistochemistry.

According to the present invention the subject prior to treatment can comprise a biological sample level MAGE-A4 and/or peptide antigen thereof of greater than or equal to 10, 25, 50, 100, 200, 300 or 400 ng/mL preferably greater than or equal to 50 ng/ml and MAGE-A4 expression is less than or equal to 1, 2, 3, 5, 7, 9% or less than 10% preferably less than or equal to 1 or 5% of cells for non-cancerous or non-tumour tissue at any intensity by immunohistochemistry.

According to the present invention the subject prior to treatment may comprise an Eastern Cooperative Oncology Group (ECOG) of 0 to 1 and/or measurable disease (gastroesophageal cancer and/or tumour) according to Response Evaluation Criteria in Solid Tumors (RECIST) 1.1 and/or histologically confirmed gastroesophageal cancer and/or tumour.

According to the present invention the subject prior to treatment is determined to be HLA-A*02 positive and/or the subject's cancer or tumour shows expression of the MAGE-A4 and/or peptide antigen thereof, for example expression of MAGE-A4 RNA or protein, preferably as herein above described.

According to the present invention if prior to treatment the subject has any one or more of:

(b) HLA-A genotype is HLA-A*02:07P as the sole HLA-A*02 allele,

(c) HLA-A genotype is HLA-A* of any A*02 null allele as the sole HLA-A*02 allele, or

(d) symptomatic CNS metastases, then the subject is not eligible for treatment.

According to the present invention the subject can be positive for HLA-A*02, for example selected from HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02:642 and/or the gastroesophageal cancer and/or tumour expresses MAGE-A4 a peptide antigen of MAGE-A4, a peptide antigen of MAGE-A4 comprising GVYDGREHTV, SEQ ID NO: 2.

According to the invention the subject can be intolerant to a standard of care treatment, preferably as described herein, additionally or alternatively the subject and/or the cancer and/or tumour can have been previously unsuccessfully treated with a standard of care treatment, for example as herein described, or been previously unsuccessfully treated with any of surgery (resection), radiation therapy, targeted therapy, immunotherapy or chemotherapy or concomitant chemotherapy with surgery (resection), radiation therapy, radiation therapy targeted therapy, or immunotherapy; or been previously unsuccessfully treated with locoregional therapy optionally selected from chemical and/or thermal percutaneous ablation and intraarterial chemoembolotherapy.

According to the present invention the cancer which is gastroesophageal cancer and/or tumour can be primary cancer, secondary cancer, relapsed cancer or refractory cancer or recurrent cancer or locally recurrent cancer, advanced or locally advanced or metastatic cancer, non-resectable cancer or locally confined, cancer with no surgical or radiotherapy option or inoperable cancer, cancer which is not amenable to transplant or loco-regional therapy or any combination thereof. The subject may have relapsed cancer or refractory cancer or recurrent cancer or locally recurrent cancer or metastatic cancer or locally confined or inoperable cancer, or any combination thereof.

Preferably the gastroesophageal cancer and/or tumour is inoperable and/or metastatic and/or advanced and/or locally advanced gastroesophageal cancer and/or tumour, for example any of cancer and/or tumour of the esophagus, gastro-esophageal junction or gastric cancer and/or tumour, preferably any of esophageal squamous-cell carcinoma (ESCC), esophageal adenocarcinoma (EAC), or esophagogastric junction cancer, carcinoma adenocarcinoma or tumour (EGJ) or stomach or gastric cancer, carcinoma or tumour, preferably inoperable or metastatic or advanced or locally advanced esophagogastric junction cancer, carcinoma adenocarcinoma or tumour (EGJ).

There is further provided the treatment method or use according to the invention wherein the subject has not received prior treatment for gastroesophageal cancer and/or tumour alternatively wherein the subject has received prior treatment for gastroesophageal cancer and/or tumour and/or has failed to respond to the prior treatment.

According to the invention the prior treatment can comprise systemic and/or local therapy, for example any one or more of; surgery, radiation therapy, cryotherapy, laser therapy, topical therapy, chemotherapy, hormonal therapy, targeted drugs, or immunotherapy. Accordingly, the prior treatment can comprise local therapy, for example any one or more of surgery, radiation therapy cryotherapy, laser therapy, topical therapy and/or systemic therapy, for example any one or more of chemotherapy, hormonal therapy, targeted drugs, or immunotherapy. According to the invention the prior treatment can comprise any one of; systemic therapy for initial diagnosis of locoregional disease, systemic therapy after diagnosis of recurrent or metastatic disease, systemic therapy after oligometastatic disease, or systemic therapy after locally recurrent disease.

Accordingly where the treatment is of gastroesophageal cancer and/or tumour the prior treatment can comprise a relevant standard of care as described herein, for treatment of primary tumor in adjuvant, locally advanced, or metastatic settings, for example which failed, for example due to progressive disease or unacceptable toxicity or intolerance. Accordingly where the treatment is of gastroesophageal cancer and/or tumour the prior treatment can comprise a systemic platinum containing chemotherapy, which may be selected from cisplatin or carboplatin chemotherapy, for treatment of primary tumor in adjuvant, locally advanced, or metastatic settings, for example which failed, for example due to progressive disease or unacceptable toxicity or intolerance. Accordingly where the treatment is of gastroesophageal cancer and/or tumour the prior treatment can comprise any of, a combined cisplatin, fluorouracil, and leucovorin or cisplatin and etoposide or carboplatin and paclitaxel, or cisplatin and capecitabine, alternatively a platinum fluoropyrimidine doublet and optionally anthracycline; an immune checkpoint inhibitor, such as pembrolizumab or nivolumab, optionally on or after platinum-based chemotherapy; a targeted therapy or targeted antibody therapy, such as trastuzumab, cetuximab, bevacizumab or ramucirumab; optionally for example which prior treatment failed, for example due to progressive disease or unacceptable toxicity or intolerance.

According to the invention the prior treatment can comprise a PD-1 axis binding antagonist, PD-L1 binding antagonist or PD-1 binding antagonist. Accordingly the prior treatment can comprise

(a) an anti-PD-L1 antibody which inhibits binding between PD-L1 and PD-1 and/or between PD-L1 and B7-1,

(b) an anti-PD-L1 antibody which inhibits PD-L1 on the cancer cell surface from transducing a signal to the intracellular pathway,

(c) an anti-PD-1 antibody which inhibits binding between PD-L1 and PD-1 and/or between PD-L2 and PD-1,

(d) an anti-PD-1 antibody which inhibits PD-1 on the T cell surface from transducing a signal to the intracellular pathway,

(e) a PD-L1 binding antagonist which is selected from;
(i) Durvalumab, Imfinzi or MED14736,
(ii) Atezolizumab, Tecentriq or MPDL3280A,
(iii) Avelumab, Bavencio or MSB0010718C,
(iv) MDX-1105, BMS-936559,

(f) a PD-1 binding antagonist is selected from;
(i) Pembrolizumab, Keytruda, Lambrolizumab or MK-3475,
(ii) Cemiplimab, Libtayo, or REGN-2810,
(iii) BMS/ONO, Nivolumab, Opdivo, ONO-4538, BMS-936558 or MDX1106.

According to the invention the prior treatment may comprise an Epidermal Growth Factor Receptor Antagonist, optionally Cetuximab. According to the invention when the prior treatment comprises chemotherapy this may comprise one or more platinum compound, optionally selected from Lipoplatin, Cisplatin, Carboplatin, Oxaliplatin, Nedaplatin, Triplatin tetranitrate, Phenanthriplatin, Satraplatin, Picoplatin. Additionally or alternatively when the prior treatment comprises chemotherapy this may comprise one or more chemotherapeutic agent selected from, methotrexate, capecitabine, taxane, anthracycline, paclitaxel, docetaxel, paclitaxel protein bound particles, doxorubicine, epirubicine, 5-fluorouracil, cyclophosphamide, afatinib, vincristine, etoposide or combinations thereof. Additionally, or alternatively when the prior treatment comprises chemotherapy this may comprise one or more chemotherapeutic agent selected from, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide. According to the invention the prior treatment can comprise any one or more of Sorafenib, a PD1 or PD-L1 antagonist or inhibitor, Regorafenib, Cabozantinib, Sunitinib, Brivanib, Everolimus, Tivantinib, Linifanib, or locoregional therapy optionally selected from chemical and/or thermal percutaneous ablation and intraarterial chemoembolotherapy, optionally for example which prior treatment failed, for example due to progressive disease or unacceptable toxicity or intolerance.

According to the invention the subject may not have received prior treatment in recurrence less than or equal to 12 months since the last treatment or less than or equal to 6 months since the last treatment. According to the invention the subject may have not received any prior adjuvant therapy (surgery followed by radiation and/or chemotherapy) in recurrence less than or equal to 12 months since the last treatment or in recurrence less than or equal to 6 months since the last treatment.

According to the invention the treatment extends or improves or effectively extends or effectively improves:

(a) progression free survival,

(b) time to progression,

(c) duration of response,

(d) overall survival,

(e) objective response or objective response rate,

(f) overall response or overall response rate,

(g) partial response or partial response rate,

(h) complete response or complete response rate;

(i) stable disease rate or median stable disease

(j) median progression free survival,

(k) median time to progression,

(l) median duration of response, or

(m) median overall survival;

(n) median objective response or median objective response rate,

(o) median overall response or median overall response rate,

(p) median partial response or median partial response rate,

(q) median complete response or median complete response,

(r) median stable disease rate or median stable disease,

in comparison to a control such as in comparison to a placebo treatment or in comparison to prior to treatment or in comparison to without treatment or in comparison to treatment comprising a standard of care as herein described.

According to the invention the treatment extends or improves or effectively extends or effectively improves any one or more of:

(a) Best Overall Response (BOR), (b) Time to Confirmed Response (TTR), (c) Duration of Response (DoR), (d) Duration of Stable Disease (DoSD), (e) Progression Free Survival (PFS), or (f) Overall Survival (OS); in comparison to a control such as in comparison to a placebo treatment or in comparison to prior to treatment or in comparison to without treatment or in comparison to treatment comprising a standard of care as herein described.

Best Overall Response (BOR), can be defined as the best response recorded from the date of T cell infusion until disease progression. Time to Confirmed Response (TTR), can be defined as the duration between T cell infusion and the initial date of the confirmed response. Duration of Response (DoR), can be defined as the duration from the initial date of the confirmed response to the date of PD, progressive disease (or death). Duration of Stable Disease (DoSD), can be defined as the duration from the date of T cell infusion to the date of PD, progressive disease (or death). Progression Free Survival (PFS), can be defined as the interval between the date T cell infusion and the earliest date of disease progression based on RECIST v1.1 or death due to any cause. Overall Survival (OS), can be defined the duration between T cell infusion and death due to any cause.

“Progression free survival” (PFS) refers to the time from treatment (or randomization) to first disease progression or death. “Time to progression” (TTP) does not count patients who die from causes other than the cancer or tumour being treated but is otherwise equivalent to PFS. “Duration of response” (DoR), is the length of time that cancer, tumour or lesion continues to respond to treatment without growing or spreading. According to the invention DoR, TTP and PFS can be assessed by Response Evaluation Criteria in Solid Tumours (RECIST) or can be assessed by CA-125 levels (cancer antigen 125) as a determinant of progression or optionally by reference to the disease biomarkers or expression of MAGE-A4, i.e. MAGE-A4 protein, peptide or mRNA in subject biological sample, cancer and/or tumour tissue or cells. Response durations, rates, readouts, measures or time points may be measured from the day on which a treatment commences for example the day on which the modified immunoresponsive cells are administered to the subject, or the day of administration of the standard of care or placebo.

According to the invention PFS and/or TTP and/or DoR, or median thereof, can be extended or improved by at least 1, 2, 3, or 4 weeks, 1 month, 2 months, 2.3 months, 2.5 months, 2.9 months, 3 months, 3.5 months, 3.8 months, 4 months, 4.5 months, 5 months, 6 months, 7 months, 8 months, 9 months, 10 months, 11 months, 12 months, 16 months, 18 months, 20 months, 22 months, 2 years, 3 years, 4 years, 5, years, 6 years, 7 years, 8 years, 9 years, 10 years in comparison to placebo treatment or in comparison to prior to treatment or in comparison to without treatment or in comparison to treatment comprising a standard of care for example as described herein (control).

In one embodiment, the PFS and/or TTP and/or DoR, or median thereof, is extended about 2.9 months to 3.8 months compared to a control. In one embodiment, the PFS and/or TTP and/or DoR, or medians thereof, is extended at least about 3.8 months compared to a control. In another embodiment, the PFS and/or TTP and/or DoR, or median thereof, is extended by about 2.3 months, in one embodiment, the PFS and/or TTP and/or DoR, or median thereof, is extended about 6 months in comparison to placebo treatment or in comparison to prior to treatment or in comparison to without treatment or in comparison to treatment comprising a standard of care for example as described herein (control).

“Overall survival” refers to a subject remaining alive for a defined period of time. According to the invention the overall survival, or median thereof, is improved or extended by about or greater than any of about 1 month, 2 months, 2.3 months, 2.5 months, 2.9 months, 3 months, 3.5 months, 3.8 months, 4 months, 4.5 months, 5 months, about 6 months, about 7 months, about 8 months, about 9 months, about 10 months, about 11 months, about 12 months, about 1.5 years, about 2 years, about 3 years, about 4 years, about 5 years, about 6 years, about 7 years, about 8 years, about 9 years, about 10 years, from initiation of the method or treatment according to the invention or from initial diagnosis, optionally the event used for survival analysis can be death from any cause. “Survival” refers to a subject remaining alive and includes progression free survival (PFS) and overall survival (OS). “Overall survival” is the length of time from either the date of diagnosis or the start of treatment for the disease, tumour and/or cancer, that subjects diagnosed with the disease are still alive. Survival can be estimated by the Kaplan-Meier method, and any differences in survival are computed using the stratified log-rank test; “extending survival” or “increasing the likelihood of survival” is meant increasing PFS and/or OS in a treated subject in comparison to placebo treatment or in comparison to prior to treatment or in comparison to without treatment or in comparison to treatment comprising a standard of care for example as described herein. According to the invention overall survival or survival can be extended or improved by at least any of about, 1 month, 2 months, 2.3 months, 2.5 months, 2.9 months, 3 months, 3.5 months, 3.8 months, 4 months, 4.5 months, 5 months, 6 months, 7 months, 8 months, 9 months, 10 months, 11 months, 12 months, 16 months, 18 months, 20 months, 22 months, 2 years, 3 years, 4 years, 5, years, 6 years, 7 years, 8 years, 9 years, 10 years in comparison to placebo treatment or in comparison to prior to treatment or in comparison to without treatment or in comparison to treatment comprising a standard of care (a control).

“Objective response rate” (ObRR) is the proportion of subjects with tumour size reduction of a predefined amount, optionally determined by sum of the longest diameters (SLD) of target lesions or tumours, and for a minimum time period. “Overall response rate (ORR)” is defined as the proportion of subjects who have a partial or complete response to therapy; it does not include stable disease. ORR is generally defined as the sum of complete responses (CR) and partial responses (PRs) over a specified time period. According to the invention ObRR and/or ORR and/or PR and/or CR and/or SD can be extended or improved by at least 5%, 10%, 15%, 20%, 25%, 30%, 35%, 40%, 45%, 50%, 55%, 60%, 65%, 70%, 75%, 80%, 85%, 90%, 95%, in comparison to placebo treatment or in comparison to prior to treatment or in comparison to without treatment or in comparison to treatment comprising a standard of care for example as described herein.

According to the present invention, the method may further comprise determining the expression level of a biomarker in a sample, biological sample, from the subject wherein the level of the biomarker is compared to a reference level in order to determine the subject's likelihood to respond to the treatment or to determine the subject's level of response to the treatment, wherein the sample is obtained either before during or after the treatment. The reference level may be the level prior to treatment of the subject or may be the level associated with the presence of cancer or the lack of presence of cancer. The biomarker may be a T-effector-associated gene, for example CD8A, perforin (PRF1), granzyme A (GZMA), granzyme B (GZMB), interferon-γ (IFN-v), CXCL9, or CXCL10. The biomarker may be an activated stroma-associated gene, for example transforming growth factor-β (TGF-β), fibroblast-activated protein (FAP), podplanin (PDPN), a collagen gene, or biglycan (BGN). The biomarker may be a or a myelokJ-derived suppressor cell-associated gene, for example CD68, CD163, FOXP3, or androgen-regulated gene 1. Alternatively, the biomarker may be PD-L1, CD8, or androgen receptor (AR) gene. Alternatively, the biomarker may be a disease biomarker as herein before described.

According to the present invention the subject undergoes lymphodepleting chemotherapy prior to administration of the modified immunoresponsive cells expressing or presenting a heterologous T-cell receptor (TCR). The lymphodepleting chemotherapy may comprise administration of cyclophosphamide and/or fludarabine. Preferably the cyclophosphamide is administered at a dose of about 100, 150, 200, 250, 300, 350, 400, 450, 500, 550, 600, 650, 700, 750, 800 or 850 mg/m²/d [d=day], preferably about 500 or 600 mg/m²/d, preferably wherein the administration is for 1 day, 2 days (×2 d), 3 days (×3 d), 4 days (×4 d) or 5 days (×5 d). Preferably the fludarabine is administered at a dose of about 5, 10, 15, 20, 25, 30, 35, 40, 450, 50, 55, 60, 65, 70, 75, 80 or 85 mg/m²/d, preferably wherein the administration is for 1 day, 2 days (×2 d), 3 days (×3 d), 4 days (×4 d) or 5 days (×5 d). Preferably the lymphodepleting chemotherapy comprises administration of cyclophosphamide and fludarabine optionally at a dose of 500 mg/m²/d×3 d cyclophosphamide and 20 mg/m2/d×3 d fludarabine or at a dose of 600 mg/m²/d×3 d cyclophosphamide and 30 mg/m2/d×4 d. According to the invention the lymphodepleting chemotherapy can administered at 3, 4, 5, 6, 7, 8, 9, 10 days preferably 7 to 5 or 7 to 4 days prior to administration of the modified immunoresponsive cells expressing or presenting a heterologous T-cell receptor (TCR). The administration of cyclophosphamide and fludarabine may be sequential separate or simultaneous, the administration may be administered intravenously or by intravenous infusion.

The invention further provides a method of

(a) reducing subject MAGE-A4 expression or concentration, for example in a subject biological sample,

(b) enhancing immune function,

(c) reducing tumour growth or tumour growth rate or maintaining tumour size after cessation of treatment or reducing tumour number or tumour burden,

(d) increasing serum cytokine and/or interferon level or concentration,

(e) improving T-cell persistence,

(f) improving T-cell infiltration of tumour,

(g) inducing a change in disease biomarker indicative of effective treatment of gastroesophageal cancer and/or tumour,

in a subject having gastroesophageal cancer and/or tumour comprising administering to the subject treatment regimen comprising an effective amount of modified immunoresponsive cells expressing or presenting a heterologous T-cell receptor (TCR) which binds MAGE-A4 or a peptide antigen of MAGE-A4 or a peptide antigen of MAGE-A4 comprising GVYDGREHTV, SEQ ID NO: 2, as herein before described with reference to the method of treatment and the aspects and embodiments and features relating thereto, optionally in comparison to prior to treatment or treatment with placebo or without treatment or treatment comprising a standard of care as described herein.

Accordingly, the invention provides a method of enhancing immune function wherein:

(a) CD8 T cells in the subject have enhanced priming, activation, proliferation and/or cytolytic activity,

(b) the number of CD8 T cells is elevated in the subject,

(c) the cancer and/or tumour cells in the subject selectively have elevated expression of MHC class I antigen expression, optionally wherein PBMC cells of the subject do not have elevated expression of MHC class I antigen,

(d) the antigen presenting cells in the subject have enhanced maturation and activation, optionally wherein the antigen presenting cells are dendritic cells,

(e) the serum levels of IL-10 and/or IL-8 in the subject are reduced,

(f) the cancer and/or tumour of the subject has elevated levels of T-cell infiltration,

(g) the T cells of the subject have reduced levels of T-cell PD-1 expression; respectively in a subject having gastroesophageal cancer and/or tumour comprising administering to the subject treatment regimen comprising an effective amount of modified immunoresponsive cells expressing or presenting a heterologous T-cell receptor (TCR) which binds MAGE-A4 or a peptide antigen of MAGE-A4 or a peptide antigen of MAGE-A4 comprising GVYDGREHTV, SEQ ID NO: 2, as herein before described with reference to the method of treatment and the aspects and embodiments and features relating thereto, optionally in comparison to prior to treatment or treatment with placebo or without treatment or treatment comprising a standard of care as described herein.

Accordingly with reference to the foregoing and the subject treated (a) the CD8 T cell activation may be characterised by an elevated frequency of gamma-IFN⁺ CD8 T cells and/or enhanced cytolytic activity; (b) the maturation of the antigen presenting cells may be characterised by increased frequency of CD83⁺ dendritic cells; (c) the activation of the antigen presenting cells may be characterised by elevated expression of CD80 and CD86 on dendritic cells; (d) the CD8 T cell may be an antigen-specific CD8 T cell.

According to the invention there is provided;

(a) a kit comprising comprising an effective amount of modified immunoresponsive cells expressing or presenting a heterologous T-cell receptor (TCR) which binds MAGE-A4 or a peptide antigen of MAGE-A4 or a peptide antigen of MAGE-A4 comprising GVYDGREHTV, SEQ ID NO: 2, and a package insert comprising instructions for using the modified immunoresponsive cells to treat or delay the progression of gastroesophageal cancer and/or tumour in a subject,

(b) a kit comprising comprising an effective amount of modified immunoresponsive cells expressing or presenting a heterologous T-cell receptor (TCR) which binds MAGE-A4 or a peptide antigen of MAGE-A4 or a peptide antigen of MAGE-A4 comprising GVYDGREHTV, SEQ ID NO: 2 and a package insert comprising instructions for using the modified immunoresponsive cells in a method of
(i) reducing subject MAGE-A4 expression or concentration, for example in a subject biological sample,
(ii) enhancing immune function,
(iii) reducing tumour growth or tumour growth rate or maintaining tumour size after cessation of treatment or reducing tumour number or tumour burden,
(iv) increasing serum cytokine and/or interferon level or concentration,
(v) improving T-cell persistence,
(vi) improving T-cell infiltration of tumour, or
(vii) inducing a change in disease biomarker indicative of effective treatment of gastroesophageal cancer and/or tumour,

in a subject having gastroesophageal cancer and/or tumour as herein before described.

The invention will be further described by reference to the following figures and examples.

### FIGURES

FIG. 1. CT scan (computed tomography scan) data demonstrating a >42% decrease in target lesions in esophagogastric junction (EGJ) cancer at a 12 week period following MAGE-A4 CD8 T-cell infusion for the cohort subject receiving around a 10 billion cell infusion.

FIG. 2. Table of tumour response for esophagogastric junction (EGJ) cancer following MAGE-A4 CD8 T-cell infusion, showing the % change reduction from baseline lesion SLC i.e. percent changes in sum of diameters in target lesions measurement (Sum of Diameters=Sum of the long diameters for non-nodal lesions and short axis for nodal lesions), responses evaluated by RECIST v1.1.

### EXAMPLES

### Example 1—a Phase I Open Label, Clinical Trial Evaluating the Safety and Anti-Tumour

Activity of Autologous T Cells Expressing Enhanced TCRs Specific for MAGE-A4, MAGE-A4 CD8 TCR in HLA-A2+ subjects with MAGE-A4 positive esophagogastric junction (EGJ) cancer, gastric cancer.

Methods

The following presents an in human study of genetically engineered ADP-A2M4CD8 SPEAR T-cells in subjects with HLA-A*02 and MAGE-A4 positive inoperable locally advanced or metastatic tumors for esophagogastric junction (EGJ) cancer, gastric cancer.

Disease was histologically or cytogenetically confirmed and/or measurable disease recorded according to RECIST v1.1 criteria. Subjects who were eligible based on HLA type and who met MAGE-A4 criteria were screened for general health, performance status and disease stage. Following Screening, subjects meeting all eligibility criteria underwent leukapheresis to obtain cells for the manufacture of autologous MAGE-A4 CD8 TCR bearing T-cells.

Eligible subjects had an ECOG Performance Status 0-1, adequate organ function and measurable disease required prior to lymphodepletion and:

(a) Subject is positive for at least one HLA-A*02 inclusion allele.

(b) Subject has inoperable or metastatic (advanced) of the esophageal (squamous or adenocarcinoma), esophagogastric Junction (EGJ), or gastric cancer.

(c) Subject may have received a fluoropyrimidine (e.g. fluorouracil or capecitabine) and/or platinum regimen

(d) Subject cancer and/or tumour may have Her2neu amplification but have failed (progressive disease or unacceptable toxicity) or refused trastuzumab.

(e) Subject may have received no more than three prior systemic regimens.

(b) Subject has histologically or cytologically confirmed diagnosis of metastatic (advanced) of the esophageal (squamous or adenocarcinoma), esophagogastric Junction (EGJ), or gastric cancer.

Exclusion of subjects is based primarily on HLA-A genotype ie if: Subject is positive for any HLA-A*02 allele other than: one of the inclusion alleles, HLA-A*02:07P or HLA-A*02 null alleles (HLA-A*02:07P or HLA-A*02 null alleles are both alleles with low activity, so if these allele's are the subject's only 02 allele then they would not be eligible). Excluded subjects additionally include those with symptomatic CNS metastases or active autoimmune or immune mediated disease, or infection.

Following leukapheresis the cells are subsequently transduced with the MAGE-A4 CD8 TCR T cells (SEQ ID NO: 5, 7+SEQ ID NO: 3) specific for MAGE-A4 antigen (particularly the specific MAGE-A4 antigenic peptide SEQ ID NO:2) and the cells expanded and cryopreserved for later use. Once the MAGE-A4 CD8 TCR T cells were available, subjects underwent lymphodepleting chemotherapy with cyclophosphamide plus fludarabine on Days −7 to −5, or Days −7 to −4 followed by infusion of transduced cells on Day 1.

Three subject cohorts were treated dosing with between 100 million to 5 billion transduced cells respectively with no dose escalation:

100 mn cell dose, (cyclophosphamide: 500 mg/m²/d)×3 d; (fludarabine: 20 mg/m²/d)×3 d

1 bn cell dose, (cyclophosphamide: 500 mg/m²/d)×3 d; (fludarabine: 20 mg/m²/d)×3 d

5 bn cell dose, (cyclophosphamide: 600 mg/m²/d)×3 d; (fludarabine: 30 mg/m²/d)×4 d

Subjects are hospitalised for 7 days following infusion and monitored for safety, T-cell persistence, cytokine production with CT and MRI performed at weeks 4, 8, 16, 24 and 3 monthly thereafter until disease progression or early interventional withdrawal, long term follow up annually is planned for a 15 year period.

A subject will be considered completing the interventional phase of the study when he/she has received T-cell infusion and then progressed or died prior to disease progression. Optionally a second T-cell infusion may be given, and they will remain in the interventional phase of the study until they have further progression of disease. Once progression is established, no further efficacy assessments are performed other than overall survival. All subjects completing from the interventional portion of the study will enter the long-term follow-up (LTFU) phase for observation of delayed adverse events (AEs) during the 15 years post-infusion in accordance with FDA and EMA regulations. This study will be considered complete when the last living subject has completed LTFU.

To evaluate the safety and tolerability of MAGE-A4 CD8 TCR T cells the incidence of dose limiting toxicities (DLTs) is monitored, determination is made of optimally tolerated dose range, adverse events (AEs), and Serious Adverse Events (SAEs); laboratory assessments, including chemistry, haematology, and coagulation; and cardiac assessments, including ECG and cardiac Troponin.

During the study MAGE-A4 is evaluated as a biomarker for tumour MAGE-A4 expression, and antitumor activity. This is performed to correlate the level of antigen expression in tumour level at Baseline, and post MAGE-A4 CD8 TCR T cell infusion. Post-therapy MAGE-A4 expression in tumour over time is assessed to determine tumour immunity or resistance to MAGE-A4 CD8 TCR T cells. Additionally, circulating cytokines were measured and evaluated for association with cytokine release syndrome (CRS) and other adverse events (AEs). Additionally post MAGE-A4 CD8 TCR T cell infusion, transduced cell persistence is assessed by determination of serum level persistence of MAGE-A4 CD8 TCR engineered T-cell as measured by MAGE-A4 TCR vector copy number and MAGE-A4 CD8 TCR transduced T-cell number. Mean expression of specific surface markers on gene-modified T cells in subject blood and tumour were measured by fluorescence intensity. Killing profile and cytokine profile of genetically modified T cells were evaluated using flow cytometry in blood and tumour. Biomarkers of subject sample including polymorphisms in cytokine genes and cytokine production.

To evaluate anti-tumour activity of MAGE-A4 CD8 TCR T cells the following endpoints are monitored by RECIST v1.1; Overall Response Rate (ORR) defined as the proportion of subjects with a confirmed complete response (CR) or partial response (PR). Additional endpoints are monitored for duration of response (DoR), duration of stable disease (SD), progression free survival (PFS), overall survival (OS). Evaluation was made of the efficacy of the treatment by assessment of duration of response and assessment of overall survival. Also assessed were intervals for

(a) the date of first T cell infusion dose and first documented evidence of CR or PR and evaluation of the efficacy of the treatment by assessment of time to first response.

(b) the date of first documented evidence of CR or PR until first documented disease progression or death due to any cause.

(c) the date of first documented evidence of stable disease (SD) until first documented disease progression or death due to any cause.

(d) the date of first T cell infusion and the earliest date of disease, progression or death due to any cause.

(e) between the date of first T cell infusion and date of death due to any cause.

Evaluation of the efficacy of the treatment by Number and % of subjects having any Long Term Follow Up Adverse Events (AEs), malignancy, neurologic disorder, rheumatologic or other autoimmune disorder, hematologic disorder, infections.

Subjects were additionally monitored for safety and tolerability response through laboratory assessments including chemistry, hematology and coagulation, and anti-MAGE-A4 TCR antibodies, adverse events (AE), including serious adverse events (SAEs), dose limiting toxicities (DLT) NCI CTCAE and optimally tolerated dose range and evaluation of persistence of genetically modified T cells in the periphery and retention of heterologous TCR expression in the T cells PBMCs using PCR-based assay.

Results

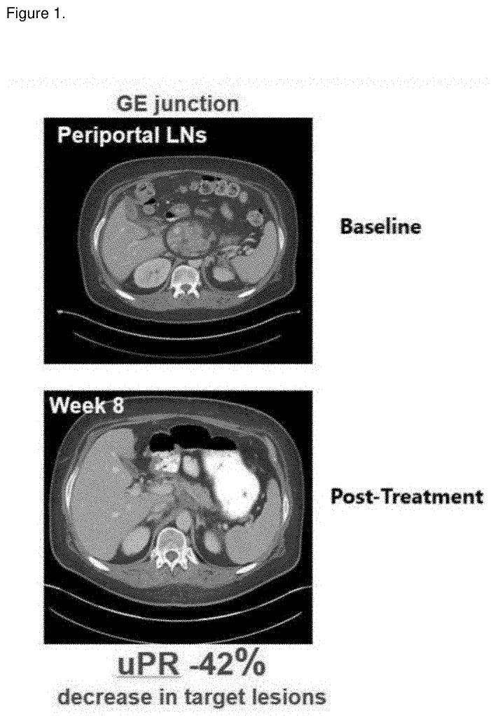

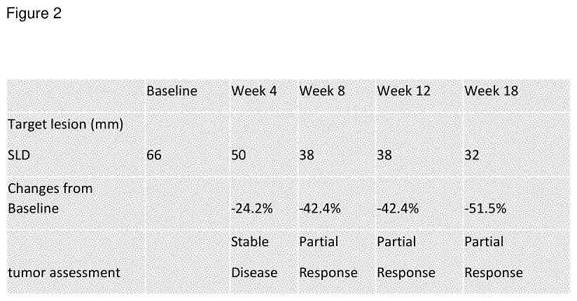

The data in FIG. 1 represent the CT scans of 31-year-old man with stage 4 adenocarcinoma of the GEJ, which is HER2 negative, the subject had received prior unsuccessful chemotherapy, targeted therapy and immunotherapy regimens (Ramucirumab+Paclitaxel, Atezolizumab+BL-8040, FLOT/FOLFIRI chemotherapy). The subject had moderate MAGE-A4 expression at baseline (IHC 3+) and large disease burden; baseline lesion SLD was 66 cm and the subject was provided a first infusion of ˜10 billion MAGE-A4 CD8 TCR T cells, adverse reaction was minimal and consistent with those typically experienced by cancer patients undergoing cytotoxic chemotherapy and/or cancer immunotherapy. The tumour showed a greater than 42% decrease at 8 weeks by RECIST 1.1 and by sum of diameters in target lesions over the measured period of weeks following the date of T cell infusion. This progressed to greater than 51% decrease at week 18 see as shown in FIG. 2. The data confirms the 18 week efficacy of response in terms of tumour size reduction (51% reduction) for esophagogastric Junction (EGJ) cancer treatment. Data is shown for aortocaval lymph nodes, equivalent data were obtained by CT for periportal lymph nodes, ascites & nonmeaurable peritoneal mets, equivalent reductions from baseline were observed as for aortocaval lymph nodes.

Over the period of 8 to 12 weeks post infusion the absolute concentration of transduced lymphocyctes comprising MAGE-A4 CD8 TCR T cells was retained at a high level (46-53%) showing durability of the therapeutic T cells.

Sequences
MAGE A4
SEQ ID NO: 1,
MSSEQKSQHC KPEEGVEAQE EALGLVGAQA PTTEEQEAAV SSSSPLVPGT
LEEVPAAESA GPPQSPQGAS ALPTTISFTC WRQPNEGSSS QEEEGPSTSP
DAESLFREAL SNKVDELAHF LLRKYRAKEL VTKAEMLERV IKNYKRCFPV
IFGKASESLK MIFGIDVKEV DPASNTYTLV TCLGLSYDGL LGNNQIFPKT
GLLIIVLGTI AMEGDSASEE EIWEELGVMG VYDGREHTVY GEPRKLLTQD
WVQENYLEYR QVPGSNPARY EFLWGPRALA ETSYVKVLEH VVRVNARVRI
AYPSLREAAL LEEEEGV
MAGE A4 peptide
SEQ ID NO: 2,
GVYDGREHTV
(CD8α)CDRs bold underlined, signal sequence italic
underlined
SEQ ID NO: 3;
MALPVTALLLPLALLLHAARPSQFRVSPLDRTWNLGETVELKCQVLLSNPTSGCSWLFQPRGAAASPT
FLLYLSQNKPKAAEGLDTQRFSGKRLGDTFVLTLSDFRRENEGYYFCSALSNSIMYFSHFVPVFLPAK
PTTTPAPRPPTPAPTIASQPLSLRPEACRPAAGGAVHTRGLDFACDIYIWAPLAGTCGVLLLSLVITL
YCNHRNRRRVCKCPRPVVKSGDKPSLSARYV
(CD8α)
SEQ ID NO: 4;
ATGGCCTTACCAGTGACCGCCTTGCTCCTGCCGCTGGCCTTGCTGCTCCACGCCGCCAGGCCGAGCCA
GTTCCGGGTGTCGCCGCTGGATCGGACCTGGAACCTGGGCGAGACAGTGGAGCTGAAGTGCCAGGTGC
TGCTGTCCAACCCGACGTCGGGCTGCTCGTGGCTCTTCCAGCCGCGCGGCGCCGCCGCCAGTCCCACC
TTCCTCCTATACCTCTCCCAAAACAAGCCCAAGGCGGCCGAGGGGCTGGACACCCAGCGGTTCTCGGG
CAAGAGGTTGGGGGACACCTTCGTCCTCACCCTGAGCGACTTCCGCCGAGAGAACGAGGGCTACTATT
TCTGCTCGGCCCTGAGCAACTCCATCATGTACTTCAGCCACTTCGTGCCGGTCTTCCTGCCAGCGAAG
CCCACCACGACGCCAGCGCCGCGACCACCAACACCGGCGCCCACCATCGCGTCGCAGCCCCTGTCCCT
GCGCCCAGAGGCGTGCCGGCCAGCGGCGGGGGGCGCAGTGCACACGAGGGGGCTGGACTTCGCCTGTG
ATATCTACATCTGGGCGCCCTTGGCCGGGACTTGTGGGGTCCTTCTCCTGTCACTGGTTATCACCCTT
TACTGCAACCACAGGAACCGAAGACGTGTTTGCAAATGTCCCCGGCCTGTGGTCAAATCGGGAGACAA
GCCCAGCCTTTCGGCGAGATACGTCGGTTCAAGAGCTAAAAGAAGTGGTAGTGGTGCCCCTGTGA
(MAGE A4 TCR α chain) CDRs bold underlined
SEQ ID NO: 5;
MKKHLTTFLVILWLYFYRGNGKNQVEQSPQSLIILEGKNCTLQCNYTVSPFSNLRWYKQDTGRGPVSL
TILTFSENTKSNGRYTATLDADTKQSSLHITASQLSDSASYICVVSGGTDSWGKLQFGAGTQVVVTPD
IQNPDPAVYQLRDSKSSDKSVCLFTDFDSQTNVSQSKDSDVYITDKTVLDMRSMDFKSNSAVAWSNKS
DFACANAFNNSIIPEDTFFPSPESSCDVKLVEKSFETDTNLNFQNLSVIGFRILLLKVAGFNLLMTLR
LWSSGSRAKR
(MAGE A4 TCR α chain coding sequence)
SEQ ID NO: 6;
ATGAAGAAGCACCTGACCACCTTTCTCGTGATCCTGTGGCTGTACTTCTACCGGGGCAACGGCAAGAA
CCAGGTGGAACAGAGCCCCCAGAGCCTGATCATCCTGGAAGGCAAGAACTGCACCCTGCAGTGCAACT
ACACCGTGTCCCCCTTCAGCAACCTGCGGTGGTACAAGCAGGACACCGGCAGAGGCCCTGTGTCCCTG
ACCATCCTGACCTTCAGCGAGAACACCAAGAGCAACGGCCGGTACACCGCCACCCTGGACGCCGATAC
AAAGCAGAGCAGCCTGCACATCACCGCCAGCCAGCTGAGCGATAGCGCCAGCTACATCTGCGTGGTGT
CCGGCGGCACAGACAGCTGGGGCAAGCTGCAGTTTGGCGCCGGAACACAGGTGGTCGTGACCCCCGAC
ATCCAGAACCCTGACCCTGCCGTGTACCAGCTGCGGGACAGCAAGAGCAGCGACAAGAGCGTGTGCCT
GTTCACCGACTTCGACAGCCAGACCAACGTGTCCCAGAGCAAGGACAGCGACGTGTACATCACCGACA
AGACCGTGCTGGACATGCGGAGCATGGACTTCAAGAGCAATAGCGCCGTGGCCTGGTCCAACAAGAGC
GACTTCGCCTGCGCCAACGCCTTCAACAACAGCATTATCCCCGAGGACACATTCTTCCCAAGCCCCGA
GAGCAGCTGCGACGTCAAGCTGGTGGAAAAGAGCTTCGAGACAGACACCAACCTGAACTTCCAGAACC
TGAGCGTGATCGGCTTCAGAATCCTGCTGCTGAAGGTGGCCGGCTTCAACCTGCTGATGACCCTGAGA
CTGTGGTCCAGCGGCAGCCGGGCCAAGAGA
(MAGE A4 TCR β chain) CDRs bold underlined
SEQ ID NO: 7;
MASLLFFCGAFYLLGTGSMDADVTQTPRNRITKTGKRIMLECSQTKGHDRMYWYRQDPGLGLRLIYYS
FDVKDINKGEISDGYSVSRQAQAKFSLSLESAIPNQTALYFCATSGQGAYEEQFFGPGTRLTVLEDLK
NVFPPEVAVFEPSEAEISHTQKATLVCLATGFYPDHVELSWWVNGKEVHSGVSTDPQPLKEQPALNDS
RYCLSSRLRVSATFWQNPRNHFRCQVQFYGLSENDEWTQDRAKPVTQIVSAEAWGRADCGFTSESYQQ
GVLSATILYEILLGKATLYAVLVSALVLMAMVKRKDSRG
(MAGE A4 TCR β chain coding sequence)
SEQ ID NO: 8;
ATGGCCAGCCTGCTGTTCTTCTGCGGCGCCTTCTACCTGCTGGGCACCGGCTCTATGGATGCCGACGT
GACCCAGACCCCCCGGAACAGAATCACCAAGACCGGCAAGCGGATCATGCTGGAATGCTCCCAGACCA
AGGGCCACGACCGGATGTACTGGTACAGACAGGACCCTGGCCTGGGCCTGCGGCTGATCTACTACAGC
TTCGACGTGAAGGACATCAACAAGGGCGAGATCAGCGACGGCTACAGCGTGTCCAGACAGGCTCAGGC
CAAGTTCAGCCTGTCCCTGGAAAGCGCCATCCCCAACCAGACCGCCCTGTACTTTTGTGCCACAAGCG
GCCAGGGCGCCTACGAGGAGCAGTTCTTTGGCCCTGGCACCCGGCTGACAGTGCTGGAAGATCTGAAG
AACGTGTTCCCCCCAGAGGTGGCCGTGTTCGAGCCTTCTGAGGCCGAAATCAGCCACACCCAGAAAGC
CACACTCGTGTGTCTGGCCACCGGCTTCTACCCCGACCACGTGGAACTGTCTTGGTGGGTCAACGGCA
AAGAGGTGCACAGCGGCGTGTCCACCGATCCCCAGCCTCTGAAAGAACAGCCCGCCCTGAACGACAGC
CGGTACTGCCTGAGCAGCAGACTGAGAGTGTCCGCCACCTTCTGGCAGAACCCCAGAAACCACTTCAG
ATGCCAGGTGCAGTTTTACGGCCTGAGCGAGAACGACGAGTGGACCCAGGACAGAGCCAAGCCCGTGA
CACAGATCGTGTCTGCCGAAGCTTGGGGGCGCGCCGATTGTGGCTTTACCAGCGAGAGCTACCAGCAG
GGCGTGCTGAGCGCCACCATCCTGTACGAGATCCTGCTGGGAAAGGCCACACTGTACGCCGTGCTGGT
GTCTGCCCTGGTGCTGATGGCCATGGTCAAGCGGAAGGACAGCCGGGGC
(MAGE A4 TCR α chain variable region)136AA - CDRs bold
underlined
SEQ ID NO: 9;
MKKHLTTFLVILWLYFYRGNGKNQVEQSPQSLIILEGKNCTLQCNYTVSPFSNLRWYKQDTGRGPVSL
TILTFSENTKSNGRYTATLDADTKQSSLHITASQLSDSASYICVVSGGTDSWGKLQFGAGTQVVVTPD
(MAGE A4 TCR β chain variable region)133AA - CDRs
bold underlined
SEQ ID NO: 10;
MASLLFFCGAFYLLGTGSMDADVTQTPRNRITKTGKRIMLECSQTKGHDRMYWYRQDPGLGLRLIYYS
FDVKDINKGEISDGYSVSRQAQAKFSLSLESAIPNQTALYFCATSGQGAYEEQFFGPGTRLTVLE
CDR1 MAGE A4 TCR α chain, (residues 48-53)
SEQ ID NO: 11;
VSPFSN
CDR2 MAGE A4 TCR α chain, (residues 71-76)
SEQ ID NO: 12;
LTFSEN
CDR3 MAGE A4 TCR α chain, (residues 111-125)
SEQ ID NO: 13;
CVVSGGTDSWGKLQF
CDR1 MAGE A4 TCR β chain, (residues 46-50)
SEQ ID NO: 14;
KGHDR
CDR2 MAGE A4 TCR β chain, (residues 68-73)
SEQ ID NO: 15;
SFDVKD
CDR3 MAGE A4 TCR β chain, (residues 110-123)
SEQ ID NO: 16;
CATSGQGAYEEQFF
CDR1 CD8α (residues 45-53)
SEQ ID NO: 17;
VLLSNPTSG
CDR2 CD8α (residues 72-79)
SEQ ID NO: 18;
YLSQNKPK
CDR3 CD8α (residues 118-123)
SEQ ID NO: 19;
LSNSIM

1. A method of treating, preventing or delaying the progression of cancer and/or tumour in a subject comprising administering to the subject a treatment regimen comprising an effective amount of modified immunoresponsive cells expressing or presenting a heterologous T-cell receptor (TCR) which binds a peptide antigen of MAGE A4 comprising GVYDGREHTV, SEQ ID NO: 2, wherein the cancer and/or tumour is gastroesophageal cancer and/or tumour.

2. (canceled)

3. The method according to claim 1, wherein
(a) the heterologous TCR binds specifically and/or selectively to the peptide antigen;
(b) the peptide antigen is associated with gastroesophageal cancer and/or tumour and/or is presented by tumour and/or cancer cell or tissue;
(c) the cancer and/or tumour is a MAGE A4 expressing cancer and/or tumour, and/or expresses MAGE A4 or peptide antigen thereof or a peptide antigen of MAGE A4 comprising GVYDGREHTV, SEQ ID NO: 2; and/or
(d) the peptide antigen is complexed with a peptide presenting molecule, optionally major histocompatibility complex (MHC) or human leukocyte antigen (HLA), optionally class I or class II, optionally wherein the peptide presenting molecule is HLA-A*02, optionally selected from HLA*02, HLA-A*02:01, HLA-A*02:02, HLA-A*02:03, HLA-A*02:04, HLA-A*02:05, HLA-A*02:06, HLA-A*02:642 or HLA-A*02:07, preferably HLA-A*02:01 or HLA-A*02.

4-7. (canceled)

8. The method of claim 1, wherein the heterologous TCR binds specifically and/or selectively to the peptide antigen and/or the peptide presenting molecule and/or complex thereof.

9. The method according to claim 1 wherein the peptide antigen is presented independently of a peptide presenting molecule.

10. The method according to claim 1, wherein the heterologous TCR comprises a TCR alpha chain variable domain and a TCR beta chain variable domain, wherein:
(i) the alpha chain variable domain comprises CDRs having the sequences
VSPFSN (αCDR1), SEQ ID NO:11 or amino acids 48-53 of SEQ ID NO:5, or sequence having at least 50% sequence identity thereto,
LTFSEN (αCDR2), SEQ ID NO:12 or amino acids 71-76 of SEQ ID NO:5, or sequence having at least 50% sequence identity thereto, and
CVVSGGTDSWGKLQF (αCDR3), SEQ ID NO:13 or amino acids 111-125 of SEQ ID NO:5, or sequence having at least 50% sequence identity thereto, and
(ii) the beta chain variable domain comprises CDRs having the sequences
KGHDR (βCDR1), SEQ ID NO:14 or amino acids 46-50 of SEQ ID NO:7, or sequence having at least 50% sequence identity thereto,
SFDVKD (βCDR2), SEQ ID NO:15 or amino acids 68-73 of SEQ ID NO:7, or sequence having at least 50% sequence identity thereto, and
CATSGQGAYEEQFF (βCDR3), SEQ ID NO:16 or amino acids 110-123 of SEQ ID NO:7 or sequence having at least 50% sequence identity thereto.

11. The method according to claim 1, wherein the heterologous TCR comprises a TCR in which
(a) the alpha chain variable domain comprises an amino acid sequence that has at least 80%, identity to SEQ ID NO:9, and/or the beta chain variable domain comprising an amino acid sequence that has at least 80% identity to SEQ ID NO:10,
(b) the alpha chain variable domain comprises an amino acid sequence comprising SEQ ID NO:9, and/or the beta chain variable domain comprises SEQ ID NO:10,
(c) the alpha chain comprises an amino acid sequence that has at least 80%, identity to SEQ ID NO:5, and/or the beta chain comprising an amino acid sequence that has at least 80% identity to SEQ ID NO:6, or
(d) the alpha chain comprises an amino acid sequence comprising SEQ ID NO:5, and/or the beta chain comprises an amino acid sequence comprising SEQ ID NO:6.

12. The method according to claim 1, wherein the modified immunoresponsive cells expressing or presenting a heterologous TCR further express or present a heterologous co-receptor, optionally wherein the heterologous co-receptor is a CD8 co-receptor, optionally wherein the heterologous CD8 co-receptor is heterodimer or homodimer, a CD8αβ heterodimer or a CD8αα homodimer, and/or the heterologous CDS co-receptor comprises any one of;
(a) a CDR 1 of at least 80% sequence identity to amino acid sequence VLLSNPTSG, SEQ ID NO: 17, CDR 2 of at least 80% sequence identity to amino acid sequence YLSQNKPK SEQ ID NO: 18 and CDR 3 of at least 80% sequence identity amino acid sequence LSNSIM SEQ ID NO:19,
(b) a CDR 1 of amino acid sequence VLLSNPTSG, SEQ ID NO:17, CDR 2 of amino acid sequence YLSQNKPK SEQ ID NO:18 and CDR 3 of amino acid sequence LSNSIM SEQ ID NO:19,
(c) an amino acid sequence having at least 80% sequence identity to amino acids number 22 to 235 of SEQ ID NO: 3, or 22 to 135 of SEQ ID NO: 3, or
(d) an amino acid sequence having 100% sequence identity to amino acids number 22 to 235 of sequence of SEQ ID NO: 3, or 22 to 135 of SEQ ID NO: 3.

13-14. (canceled)

15. The method according to claim 1, wherein the modified immunoresponsive cells expressing or presenting a heterologous TCR further express or present a heterologous co-stimulatory ligand, optionally 4-1BBL or CD80.

16. The method of claim 1, wherein the modified immunoresponsive cells are (a) B cells, T cells or natural killer (NK) cells, (b) T cells, optionally CD4⁺ T cells and/or CD8⁺ T cells, or (c) a population of CD4+ T cells; or CD8+ T cells, or a mixed population of CD4+ T cells and CD8+ T cells.

17. (canceled)

18. The method of claim 1, wherein the modified immunoresponsive cells are administered continuously or intermittently.

19. The method of claim 1, wherein the modified immunoresponsive cells are administered as multiple doses or is administered as a single dose, optionally wherein the single or multiple doses are administered in one or more dosing cycles, optionally wherein the dose may be a fixed dose or a variable dose.

20. (canceled)

21. The method according to claim 1 wherein the the modified immunoresponsive cells are administered at a dose of between about 500 million to about 1 billion cells, about 2 billion to about 5 billion cells or about 6 billion to about 10 billion cells.

22. The method according to claim 1 wherein the modified immunoresponsive cells are administered as;
(a) a single dose in each of one or more dosing cycles,
(b) one or more doses in each of one or more dosing cycles,
(c) a single dose on the first day of each of one or more dosing cycles,
(d) one or more doses in each of one or more dosing cycles, at least one dose being on the first day of each cycle,
(e) one or more doses in each of one or more dosing cycles, at least one dose being on the first day of each cycle,
(f) a single dose.

23-28. (canceled)

29. The method according to claim 1 wherein, the subject is intolerant to a standard of care treatment, optionally systemic platinum-based chemotherapy treatment.

30. The method according to claim 1 wherein the gastroesophageal cancer and/or tumour has been previously unsuccessfully treated with a standard of care treatment, optionally systemic platinum-based chemotherapy treatment, or previously unsuccessfully treated with any of surgery (resection), radiation therapy, targeted therapy, immunotherapy or chemotherapy or concomitant chemotherapy with surgery (resection), radiation therapy, radiation therapy targeted therapy, checkpoint inhibitor or immunotherapy.

31. (canceled)

32. The method according to claim 1 wherein the subject has or wherein the gastroesophageal cancer and/or tumour is; primary cancer, secondary cancer, relapsed cancer or refractory cancer or recurrent cancer or locally recurrent cancer or metastatic cancer, non-resectable cancer or locally confined, cancer with no surgical or radiotherapy option or inoperable cancer optionally wherein the cancer is not amenable to transplant or loco-regional therapy, and/or wherein the gastroesophageal cancer and/or tumour is any of esophageal squamous-cell carcinoma (ESCC), esophageal adenocarcinoma (EAC), esophagogastric junction cancer, carcinoma adenocarcinoma or tumour (EGJ) or stomach or gastric cancer, carcinoma or tumour, optionally metastatic and/or advanced and/or locally advanced and/or recurrent ESCC, EAC, EGJ or stomach or gastric cancer, carcinoma or tumour.

33. (canceled)

34. The method according to claim 1 wherein prior to administration of the modified immunoresponsive cells expressing or presenting a heterologous T-cell receptor (TCR) the subject undergoes lymphodepleting chemotherapy, optionally wherein the lymphodepleting chemotherapy comprises administration of cyclophosphamide and fludarabine optionally at a dose of 500 mg/m2/d×3 d cyclophosphamide and 20 mg/m2/d×3 d fludarabine or at a dose of 600 mg/m2/d×3 d cyclophosphamide and 30 mg/m2/d×4 d, and/or the lymphodepleting chemotherapy is administered 7 to 5 or 7 to 4 days prior to administration of the modified immunoresponsive cells expressing or presenting a heterologous T-cell receptor (TCR).

35-36. (canceled)

37. The method according to claim 1 wherein the subject has not received prior treatment for cancer and/or tumour.

38. The method according to claim 1, wherein the subject has received prior cancer and/or tumour treatment and/or has failed to respond to prior cancer and/or tumour treatment, optionally wherein the prior treatment is for gastroesophageal cancer and/or tumour, optionally wherein the prior treatment comprises systemic and/or local therapy, optionally any one or more of surgery, radiation therapy cryotherapy, laser therapy, topical therapy and/or systemic therapy, for example any one or more of chemotherapy, hormonal therapy, targeted drugs, targeted chemotherapy, or immunotherapy, wherein the prior treatment optionally comprises:
(a) a PD-L1 binding antagonist or PD-1 binding antagonist, optionally wherein the PD-1 axis binding antagonist or PD-L1 binding antagonist is an antibody;
(b) an Epidermal Growth Factor Receptor Antagonist, optionally any of Cetuximab, erlotinib, gefitinib or afatinib or a vascular endothelial growth factor (VEGF) inhibitor such as for example ramucirumab or bevacizumab or an EGFR inhibitor antibody, such as panitumumab or cetuximab or a human hepatocyte growth factor HGF and/or Met inhibitor, such as onartuzumab or rilotumumab;
(c) chemotherapy comprising a platinum compound, optionally selected from any of Lipoplatin, Cisplatin, Carboplatin, Oxaliplatin, Nedaplatin, Triplatin tetranitrate, Phenanthriplatin, Satraplatin, Picoplatin;
(d) chemotherapy comprising a chemotherapeutic agent selected from any of, methotrexate, capecitabine, taxane, anthracycline, paclitaxel, docetaxel, paclitaxel protein bound particles, doxorubicine, epirubicine, 5-fluorouracil, cyclophosphamide, afatinib, vincristine, etoposide or combinations thereof; or
(e) chemotherapy comprising a chemotherapeutic agent selected from any of, FEC: 5-fluorouracil, epirubicine, cyclophosphamide; FAC: 5-fluorouracil, doxorubicine, cyclophosphamide; AC: doxorubicine, cyclophosphamide; EC: epirubicine, cyclophosphamide.

39-46. (canceled)

47. The method according to claim 1 wherein the treatment effectively extends or improves:
(a) progression free survival,
(b) time to progression,
(c) duration of response,
(d) overall survival,
(e) objective response or objective response rate,
(f) overall response or overall response rate,
(g) partial response or partial response rate,
(h) complete response or complete response rate;
(i) stable disease rate or median stable disease
(j) median progression free survival,
(k) median time to progression,
(l) median duration of response,
(m) median overall survival;
(n) median objective response or median objective response rate,
(o) median overall response or median overall response rate,
(p) median partial response or median partial response rate,
(q) median complete response or median complete response, or
(r) median stable disease rate or median stable disease,
in comparison to a placebo treatment or in comparison to prior to treatment or in comparison to without treatment or in comparison to treatment comprising a standard of care, optionally systemic platinum-based chemotherapy treatment.


SEQUENCE LISTING (19 sequences)

SEQ ID NO: 1 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 2 - protein, 10 aa, Homo sapiens
GVYDGREHTV

SEQ ID NO: 3 - protein, 235 aa, Artificial Sequence
  source: /note=“Description of Artificial Sequence Synthetic polypeptide”
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 4 - DNA, 745 bp, Artificial Sequence
  source: /note=“Description of Artificial Sequence Synthetic polynucleotide”
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcggttcaagagctaaaagaagtggtagtggtgcccctgtga

SEQ ID NO: 5 - protein, 282 aa, Artificial Sequence
  source: /note=“Description of Artificial Sequence Synthetic polypeptide”
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 6 - DNA, 846 bp, Artificial Sequence
  source: /note=“Description of Artificial Sequence Synthetic polynucleotide”
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagaga

SEQ ID NO: 7 - protein, 311 aa, Artificial Sequence
  source: /note=“Description of Artificial Sequence Synthetic polypeptide”
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 8 - DNA, 933 bp, Artificial Sequence
  source: /note=“Description of Artificial Sequence Synthetic polynucleotide”
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 9 - protein, 136 aa, Artificial Sequence
  source: /note=“Description of Artificial Sequence Synthetic polypeptide”
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPD

SEQ ID NO: 10 - protein, 133 aa, Artificial Sequence
  source: /note=“Description of Artificial Sequence Synthetic polypeptide”
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLE

SEQ ID NO: 11 - protein, 6 aa, Artificial Sequence
  source: /note=“Description of Artificial Sequence Synthetic peptide”
VSPFSN

SEQ ID NO: 12 - protein, 6 aa, Artificial Sequence
  source: /note=“Description of Artificial Sequence Synthetic peptide”
LTFSEN

SEQ ID NO: 13 - protein, 15 aa, Artificial Sequence
  source: /note=“Description of Artificial Sequence Synthetic peptide”
CVVSGGTDSWGKLQF

SEQ ID NO: 14 - protein, 5 aa, Artificial Sequence
  source: /note=“Description of Artificial Sequence Synthetic peptide”
KGHDR

SEQ ID NO: 15 - protein, 6 aa, Artificial Sequence
  source: /note=“Description of Artificial Sequence Synthetic peptide”
SFDVKD

SEQ ID NO: 16 - protein, 14 aa, Artificial Sequence
  source: /note=“Description of Artificial Sequence Synthetic peptide”
CATSGQGAYEEQFF

SEQ ID NO: 17 - protein, 9 aa, Artificial Sequence
  source: /note=“Description of Artificial Sequence Synthetic peptide”
VLLSNPTSG

SEQ ID NO: 18 - protein, 8 aa, Artificial Sequence
  source: /note=“Description of Artificial Sequence Synthetic peptide”
YLSQNKPK

SEQ ID NO: 19 - protein, 6 aa, Artificial Sequence
  source: /note=“Description of Artificial Sequence Synthetic peptide”
LSNSIM